(19) United States
(12) Patent Application Publication
(10) Pub. No.: US 2023/0268037 A1
(43) Pub. Date: Aug. 24, 2023

(54) MANAGING REMOTE SESSIONS FOR USERS BY DYNAMICALLY CONFIGURING USER INTERFACES

(71) Applicant: Click Therapeutics, Inc., New York, NY (US)

(72) Inventor: Jacqueline Lutz, Cambridge, MA (US)

(73) Assignee: Click Therapeutics, Inc., New York, NY (US)

(21) Appl. No.: 18/111,084

(22) Filed: Feb. 17, 2023

Related U.S. Application Data
(60) Provisional application No. 63/312,379, filed on Feb. 21, 2022.

(51) Int. Cl. G16H 10/20; G06F 3/04842; G16H 10/60

(52) U.S. Cl. CPC G16H 10/20; G06F 3/04842; G16H 10/60; G06F 3/04847

(57) ABSTRACT

The present disclosure relates to systems and methods of performing session trials. A computing system may select, for a session trial for subject, (i) a first image from a plurality of images of expressions associated with a subject, (ii) a second image associated with another subject, and (iii) a third image corresponding to one of a plurality of types for the condition. The computing system may provide, for presentation of the session trial to the subject, (i) the first image, (ii) the second image, and (iii) the third image, in accordance with a presentation parameter. The computing system may receive, from the subject, a response identifying an association of the third image with one of the first image or the second image. The computing system may determine a performance metric based on the association. The computing system may update, using the performance metric, the presentation parameter.

### CROSS REFERENCE TO RELATED APPLICATIONS

The present application claims priority under 35 U.S.C. § 119(e) to U.S. Provisional Application No. 63/312,379, filed Feb. 21, 2022, which is incorporated herein by reference in its entirety.

### BACKGROUND

A computing device may present a stimulus in the form of an image, video, or audio to a user. In response to the presentation of the stimulus, the user may react or respond by performing an action. The computing device may record and store the user's response.

### SUMMARY

Chronic pain (e.g., pain lasting beyond the ordinary duration of healing), represents a major public health issue that affects 10%-20% of the adult general population in the European Union (EU) and United States of America (USA). As opposed to acute pain (e.g., an adaptive sensory perception to prevent injury or support healing), chronic pain can severely interfere with an individual's physiological and psychological functioning. For instance, chronic pain can impair the sense of self, and in particular, can lead to a strong association between the self-schema and pain condition (also referred herein as “self-pain enmeshment”).

Chronic pain may generally be understood in a biopsychosocial framework, exemplifying that the experience of and response to pain results from a complex interaction of biological, psychological, and social factors. Especially when pain is protracted, the influence of psychological factors (e.g., both affective and cognitive) may become more predominant. For example, many patients may develop anxiety and catastrophic thoughts regarding their pain, and pain-related rumination and depressive symptoms may be common psychopathological problems.

One psychological factor influenced by the frequent or continued experience of pain, may be the concept and evaluation of the self (i.e., self-related processes). Individuals suffering from chronic pain may experience changes in the evaluation and the description of the self. The former may demonstrated to result in increased negative self-evaluations by patients with chronic pain, including guilt and shame related to the chronic pain interfering with functioning. In other words, the repeated interference of pain with daily functioning can strengthen the association between a person's self-concept and their pain diagnosis (e.g., self-pain enmeshment). Furthermore, enmeshment may entail the incorporation of the self- and pain-schema, resulting from the repeated simultaneous activation of their elements.

Self-pain enmeshment may also relate to increased pain sensitivity and lower pain acceptance, even when controlled for depressive symptoms. Under the enmeshment theory, self-pain enmeshment may underlie cognitive biases in memory and attention that have been demonstrated in patients with chronic pain, and can therefore be assessed with implicit measures, such as the implicit association task (IAT). The IAT may be used to measure the strength of a person's automatic association between different mental concepts based on the reaction time to varying response-mappings of these concepts. These concepts may be embodied using various types of stimuli to the subject, such as visual, audio, or other sensory triggers, or any combination thereof. The IAT may be used to train stronger self-pain enmeshment in patients with chronic pain compared to healthy controls, and may demonstrate improvements in self-pain enmeshment as measured with the IAT after psychotherapy.

One approach in measuring subject's responses to IAT may be to use a computer platform along with a human assistant (e.g., clinician) to physically guide a subject through the task. There may be, however, many drawbacks and impairments with this approach. For one, the computer storing and maintaining the subject data and the stimuli for running the IAT may be accessed in one location (e.g., a laboratory or clinic). The instructions for running the IAT may also be provided in part by the human assistant. This may result in the inability to access the IAT program itself from other sites and different computers, thereby significantly limiting the ability to run IAT sessions to individual sites.

For another, the computer platform may be unable to adaptively and selectively provide stimuli tailored to a particular subject for the IAT, because these platforms may not factor in the subject's responses in near-real-time and in an objective manner. Because of this, the platform may provide stimuli that may not be relevant to the subject's mental associations with the condition to be addressed (e.g., chronic pain). Therefore, whatever responses taken from the subject via the IAT sessions may not be useful in determining the subject's mental associations. As a result, the subject may be put through (e.g., by a clinician) multiple, repeated IAT sessions on the computer platform until useful results are obtained, if ever. These repeated sessions may lead to additional consumption of computer resources, such as processor, memory, and power. Furthermore, the inability to adaptively select stimuli may lead to degradation in the quality of human-computer interactions (HCI) between the subject and the computer platform providing the IAT, with the subject being provided irrelevant stimuli with results of little use.

To address these and other technical challenges, a session management service may generate a session trial package identifying a set of images and trial parameters according to which individual IAT session trials are to be run. The service may provide the session package to an application running on an end-user device (e.g., smartphone, tablet, laptop, or desktop) to run the IAT session trials for the subject. A database accessible by the service may be used to store and maintain a set of user profiles for a respective set of subjects and a set of images of expressions. The user profile data may identify a condition of a subject to be addressed (e.g., chronic pain) and may be used to keep track of the progress of the subject throughout the IAT session trials. The images of expressions may include images of facial expressions (e.g., such as in pain or relaxed) from the subject and others with various labeled intensities.

For each session trial for a particular subject, the service may select a first image (sometimes referred to herein as a “self image”) from the set of images from the subject and a second image (sometimes referred herein as an “other image”) from the set of image from others besides the subject. In addition, the service may select a third image (sometimes herein referred as a “stimulus image”) based on the condition of the subject to be addressed and the progress of the subject as identified in the profile. The third image may be obtained from the set of images of expressions from others. Depending on the task that the subject is expected to perform for the session trial, the third image may correspond to the condition. When the third image is of an associative type, the subject may be expected to associate the third image with the first image and away from the second image. Conversely, when the third image is of a non-associative type, the subject may be expected to associate the third image with the second image and away from the first image.

With the selection, the service may determine presentation parameters for the session trial. The presentation parameters may define various specifications for the session trial. For example, the parameters may specify any or all of the following: locations for display of the first and second images within a user interface on the application of the end-user device; a location of the third image relative to the first and second images; render sizes for the first, second, and third images; start and end times for displaying of the first image and second images; and a start and end time for the third image relative to the respective times for the first and second images, among others. The presentation parameters may be determined based on the subject profile, such as measured progress and preferences, among others. With the determination, the service may include the selected images and the presentation parameters in a session trial package and provide the package to the end-user computing device of the subject.

Upon receipt, the application running on the end-user computing device may present the session trial in accordance with the specifications of the presentation parameters. For instance, the application may start displaying the first and second images in the specified locations of the user interface at the specified time. The application may then display the third image in the defined location relative to the first and second images in the user interface starting at the specified time. In conjunction, the application may generate and present one or more user interface elements (e.g., a slide bar or command buttons) to accept the subject's response. Using the user interface elements, the subject may input the response to indicate an association of the third image with one of the first image or the second image. The measured response may also identify the subject's response time corresponding to a time elapsed between the initial display of the third image with the inputting of the response. The application may send the subject's response to the service.

Based on the subject's response for the session trial, the service may determine a performance metric of the subject. The performance metric may identify whether the subject performed the task of the trial correctly, and by extension may measure an amount of mental association between the subject himself or herself and the condition. When the third image is of the associative type and the response indicates an association between the second image and the third image, the performance metric may indicate a correct association. Otherwise, when the third image is of the associative type and the response indicates an association between the second image and the third image, the performance metric may indicate an incorrect association. Likewise, when the third image is of the non-associative type and the response indicates an association between the first image and the third image, the performance metric may indicate a correct association. When the third image is of the non-associative type and the response indicates an association between the first image and the third image, the performance metric may indicate an incorrect association.

Using the performance metric, the service may update the subject profile data to indicate the progress of the subject with respect to the condition. The service may also modify the presentation parameters for the next session trial. For example, when the performance metric decreases, the service may increase the amount of time the third image is displayed or may enlarge the distance between the third image and the first image and the second image. When the performance metric increases, the service may select images of expression with lower intensity for the condition. The selection of images may be based on a defined function of the performance metric and the subject's progress. The service may store and maintain the performance metric along with the subject profile data.

In this manner, the session management service may provide the ability to run the IAT with the capability of providing stimuli images across a wide assortment of platforms outside the confines of a location such as the laboratory or clinic. This may greatly improve the overall utility of the application providing the IAT session. In addition, by incorporating subject response data, the service may dynamically update the presentation parameters and adaptively select stimuli images in an objective manner to provide session packages. The updating of the parameters and adaptive selection of images may reduce or eliminate the instances of multiple repeated trials with non-useful results, thereby increasing efficiency and saving consumption of computer resources (e.g., the processor, memory, and power). The session package may also increase the quality of HCI between the subject and the overall system, including with the user interface of the application providing the IAT.

Aspects of the present disclosure are directed to systems, methods, and non-transitory computer readable media for managing sessions for subjects. A computing system may have one or more processors coupled with memory. The computing system may identify, using a user profile of a subject maintained on a database, a condition of the subject to be addressed and a plurality of images of expressions associated with the subject. The computing system may select, for a first session trial for subject, (i) a first image from the plurality of images of expressions associated with the subject, (ii) a second image associated with another subject, and (iii) a third image corresponding to one of a plurality of types for the condition. The computing system may determine a presentation parameter for the first session trial based on the user profile. The computing system may provide, for presentation of the first session trial to the subject, (i) the first image, (ii) the second image, and (iii) the third image, in accordance with the presentation parameter. The computing system may receive, from the subject, a response identifying an association of the third image with one of the first image or the second image. The computing system may determine a performance metric of the subject for the first session trial based on the association identified in the response and a type of the plurality of types corresponding to the third image. The computing system may update, using the performance metric, the presentation parameter to modify the presentation for a second session trial and the user profile in relation to the condition.

In some embodiments, the computing system may select the third image corresponding to an associative type for the condition. In some embodiments, the computing system may determine, responsive to the association of the third image as with the second image, the performance metric to indicate the response as a correct selection.

In some embodiments, the computing system may select the third image corresponding to an associative type for the condition. In some embodiments, the computing system may determine, responsive to the association of the third image as with the first image, the performance metric to indicate the response as an incorrect selection.

In some embodiments, the computing system may select, from a plurality of images of expressions associated with one or more subjects, the third image based on an intensity level for the first session trial. In some embodiments, the computing system may determine the presentation parameter to define a length of the presentation of the third image on a display, using a second performance metric of a third session trial.

In some embodiments, the computing system may determine the presentation parameter to define a location of the presentation of the third image on a display relative to the first image and the second image to increase likelihood of a correct selection. In some embodiments, the computing system may determine the performance metric based on a comparison between (i) a time elapsed between the presentation of the third image and the receipt of the response and (ii) a threshold time for the third image.

In some embodiments, the computing system may provide, responsive to receiving the response, for presentation to the subject, an indication of the response as one of a correct selection or an incorrect selection based on the association. In some embodiments, the computing system may provide, via a display, a graphical user interface to associate the third image with one of the first image or the second image.

In some embodiments, the condition may be a condition associated with chronic pain. In some embodiments, the condition associated with chronic pain may include one or more of the following: arthritis, migraine, fibromyalgia, back pain, Lyme disease, endometriosis, repetitive stress injuries, irritable bowel syndrome, inflammatory bowel disease or cancer pain.

### BRIEF DESCRIPTION OF THE DRAWINGS

The foregoing and other objects, aspects, features, and advantages of the disclosure will become more apparent and be better understood by referring to the following descriptions taken in conjunction with the accompanying drawings, in which:

FIG. 1 depicts a block diagram of a system for managing sessions for subjects to acquire response data, in accordance with an illustrative embodiment;

FIG. 2 depicts a block diagram of a process of providing session packages in the system for managing sessions, in accordance with an illustrative embodiment;

FIG. 3 depicts a block diagram of an example user interface for a session presented to subjects, in accordance with an illustrative embodiment;

FIG. 4 depicts a block diagram of a process of recording subject responses in the system for managing sessions, in accordance with an illustrative embodiment;

FIG. 5 depicts a flow diagram of a method of performing sessions for users to acquire response data, in accordance with an illustrative embodiment;

FIG. 6A depicts a flow diagram of a method of running trial sessions, in accordance with an illustrative embodiment;

FIG. 6B depicts a flow diagram of an embodiment of selection status determination, in accordance with an illustrative embodiment;

FIG. 6C depicts a flow diagram of an embodiment of stimulus threshold determination and stimulus selection, in accordance with an illustrative embodiment;

FIG. 6D depicts a flow diagram of an embodiment of stimulus display period determination diagram, in accordance with an illustrative embodiment;

FIG. 7 depicts a block diagram of a process for providing treatment regimen in the system for managing sessions, in accordance with an illustrative embodiment; and

FIG. 8 is a block diagram of a server system and a client computer system in accordance with an illustrative embodiment.

### DETAILED DESCRIPTION

For purposes of reading the description of the various embodiments below, the following enumeration of the sections of the specification and their respective contents may be helpful:

Section A describes systems and methods for managing remote session trials for subjects; and

Section B describes a network and computing environment which may be useful for practicing embodiments described herein.

### A. Systems and Methods for Managing Remote Session Trials for Subjects

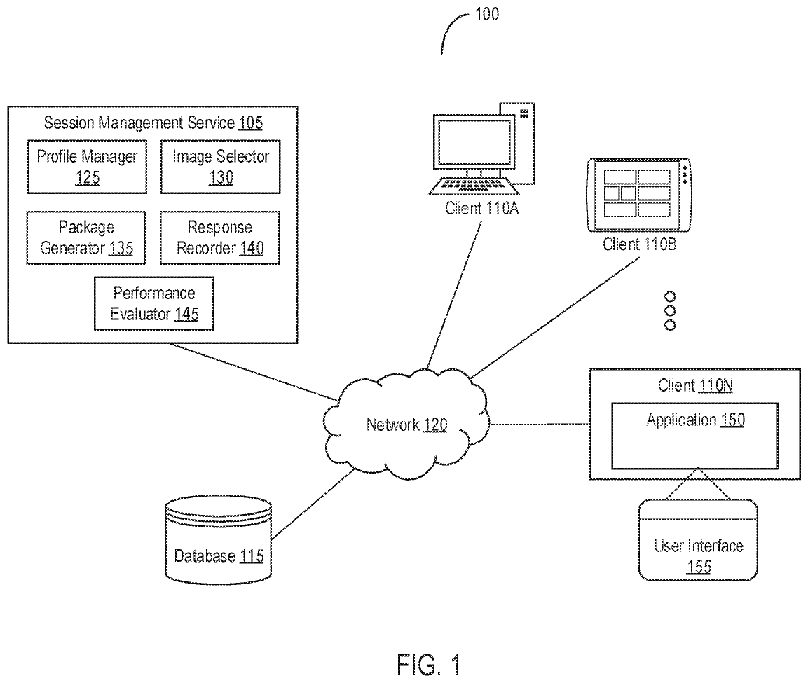

Referring now to FIG. 1, among others, depicted is a block diagram of a system 100 for managing sessions for subjects to acquire response data. In overview, the system 100 may include at least one session management service 105, one or more clients 110A-N (hereinafter generally referred to clients 110), and at least one database 115, communicatively coupled with one another via at least one network 120. The session management service 105 may include at least one profile manager 125, at least one image selector 130, at least one package generator 135, at least one response recorder 140, and at least one performance evaluator 145, among others. At least one client 110 (e.g., the client 110N as depicted) may include at least one application 150 to provide at least one user interface 155.

Each of the components in the system 100 (e.g., the session management service 105 and its components, the client 110 and its components, the database 115, and the network 120) may be executed, processed, or implemented using hardware or a combination of hardware, such as the system 800 detailed herein in Section B.

In further detail, the session management service 105 (sometimes herein generally referred to as a computing system or a service) may be any computing device comprising one or more processors coupled with memory and software and capable of performing the various processes and tasks described herein. The session management service 105 may be in communication with the one or more clients 110 and the database 115 via the network 120. The session management service 105 may be situated, located, or otherwise associated with at least one server group. The server group may correspond to a data center, a branch office, or a site at which one or more servers corresponding to the session management service 105 is situated.

Within the session management service 105, the profile manager 125 may store, maintain, and update data associated users of instances of the application 150 accessed on the respective clients 110. The image selector 130 may identify a set of images to provide to the user as part of a session trial. The package generator 135 may create session packages including the set of images and trial presentation parameters to provide to the application 150 accessed on the client 110. The response recorder 140 may retrieve user responses from the application 150 on the clients 110. The performance evaluator 145 may evaluate the user responses to update the data associated with the users and to update the trial presentation parameters.

The client 110 (sometimes herein referred to as an end user computing device) may be any computing device comprising one or more processors coupled with memory and software and capable of performing the various processes and tasks described herein. The client 110 may be in communication with the session management service 105 and the database 115 via the network 120. The client 110 may be situated, located, or otherwise positioned at any location, independent of the session management service 105 or any medical facility. For instance, the client 110 may be a smartphone, other mobile phone, tablet computer, wearable computing device (e.g., smart watch, eyeglasses), or laptop computer that can carried around by a user. The client 110 may be used to access the application 150. In some embodiments, the application 150 may be downloaded and installed on the client 110 (e.g., via a digital distribution platform). In some embodiments, the application 150 may be a web application with resources accessible via the network 120.

The application 150 on the client 110 may be a digital therapeutics application and may provide a session (sometimes referred to herein as a therapy session) via the user interface 150 to address at least one condition of the user (sometimes herein referred to as a patient, person, or subject). The condition may include, for example, a chronic pain disorder. The chronic pain may be associated with or include arthritis, migraine, fibromyalgia, back pain, Lyme disease, endometriosis, repetitive stress injuries, irritable bowel syndrome, inflammatory bowel disease, and cancer pain, among others. The session provided via the application 150 may include a set of session trials with tasks to be performed by the user. The user may be at least partially concurrently taking medication to address the chronic pain or the associated condition, while being provided session trials through application 150. The medication may include, for example: acetaminophen; a nonsteroidal anti-inflammatory composition (e.g., aspirin, ibuprofen, and naproxen); an antidepressant (e.g., tricyclic antidepressant such as amitriptyline, imipramine, nortriptyline, doxepin; selective serotonin or norepinephrine reuptakes inhibitors, such as duloxetine; or selective serotonin reuptake inhibitors, such as fluoxetine, paroxetine, or sertraline); an anticonvulsant (e.g., carbamazepine, gabapentin, or pregabalin); or other composition (e.g., triptans, antiemetics, ergots, neurotoxin injections, calcitonin gene-related peptide (CGRP) inhibitors, beta-blockers, or anti-epileptic); among others. The user of the application 150 may also other psychotherapies for these conditions.

The database 115 may store and maintain various resources and data associated with the session management service 105 and the application 150. The database 115 may include a database management system (DBMS) to arrange and organize the data maintained thereon. The database 115 may be in communication with the session management service 105 and the one or more clients 110 via the network 120. While running various operations, the session management service 105 and the application 150 may access the database 115 to retrieve identified data therefrom. The session management service 105 and the application 150 may also write data onto the database 115 from running such operations.

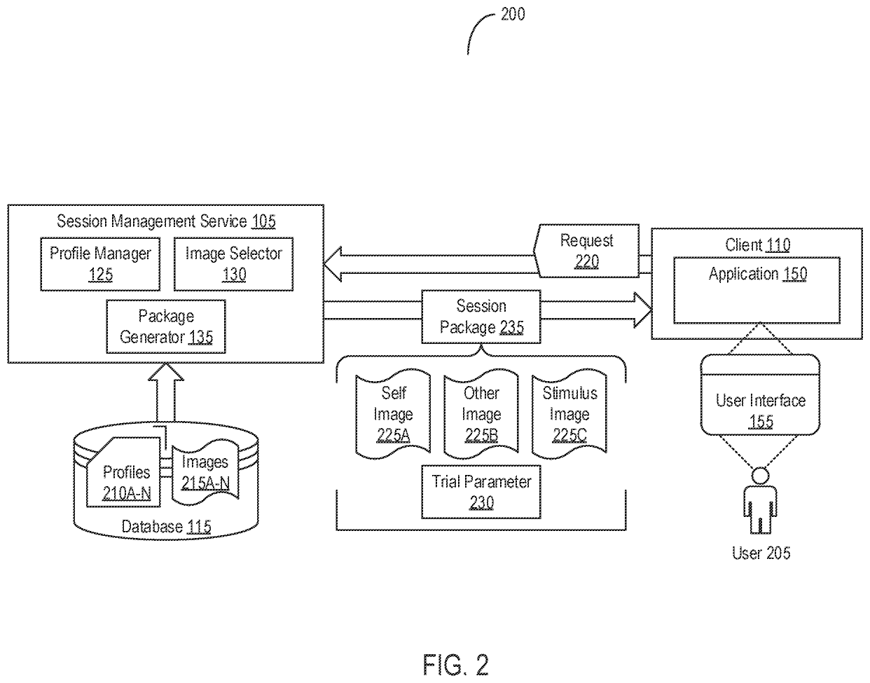

Referring now to FIG. 2, among others, depicted is a block diagram of a process 200 of providing session packages in the system 100 for managing sessions. The process 200 may include or correspond to operations in the system 100 to generate session packages to define session trials to provide to a user 205 of the application 150 on the client 110. Under the process 200, the profile manager 125 executing on the session management service 105 may handle, administer, or otherwise manage a set of user profiles 210A-N (hereinafter generally referred as user profiles 210) in the database 115. The profile manager 125 may generate each user profile 210 in the database 115 as at least partially part of the registration of the user 205 with the session management service 105 for using the application 150. For instance, when first accessing the application 150, the user 205 may have previously registered with the session management service 105 and provided a user identifier (e.g., an account identifier or name) and the condition to be addressed.

Each user profile 210 (sometimes herein referred to as a subject profile) may be associated with or correspond to a respective user 205 of the application 150. The user profile 210 may identify include various information about the user 205, such as a user identifier, the condition to be addressed (e.g., chronic pain or associated ailments), information on session trials carried out by the user 205, a performance metric across session trials, and progress in addressing the condition, among others. The information on session trials may include various parameters of previous session trials performed by the user 205, and may be initially be null. The performance metric may be initially set to a start value (e.g., null or “0”) and may indicate or correspond to an ability to the user 205 to correctly and quickly perform the tasks via the user interface 155 of the application 150. The progress may also initially be set to a start value (e.g., null or “0”) and may correspond to alleviation, relief, or treatment of the condition. In some embodiments, the user profile 210 may identify or include information on treatment regimen undertaken by the user 205, such as type of treatment (e.g., therapy, pharmaceutical, or psychotherapy), duration (e.g., days, weeks, or years), and frequency (e.g., daily, weekly, quarterly, annually), among others. The user profile 210 may be stored and maintained in the database 115 using one or more files (e.g., extensible markup language (XML), comma-separated values (CSV) delimited text files, or a structured query language (SQL) file). The user profile 210 may be iteratively updated as the user 205 performs additional session trials.

In addition, the profile manager 125 may handle, administer, or manage a set of images 215A-N (hereinafter generally referred to as images 215) in the database 115. The images 215 may be of facial expressions from various human subjects. Each image 215 may be associated with an annotation identifying a type of facial expression within the image 215 and an intensity level for the facial expression. The expression may correspond to the condition or the lack thereof. For example, for the condition of chronic pain, the image 215 may show facial expressions of angst or pain (e.g., affected by chronic pain) or happy or relaxed (e.g., disassociated with chronic pain). The intensity level may correspond to a degree to which the facial expression in the image 215 is affected by the condition. For instance, an image 215 with a high intensity level may show a facial expression in extreme pain, whereas an image 215 with a low intensity level may depict a relaxed facial expression. In some embodiments, other forms of stimuli may be used instead of or in addition to images 215, such as text, audio, or haptic stimuli, among others.

At least a portion of the images 215 may be aggregated by the profile manager 125 from users 205 of the application 150. For example, in registering with the session management service 105, the application 150 may acquire one or more images of facial expressions of the user 205 via a camera of the client 110. For each image, the application 150 may prompt the user 205 for a specified type of facial expression (e.g., grimacing, sad, happy, or relaxed) at a certain intensity level. The application 150 may send the images with an annotation identifying the corresponding types of facial expressions and intensities, together with the user identifier for the user 205, to the session management service 105. With receipt, the profile manager 125 may store and maintain the images as part of the overall set of images 215 in the database 115. The images 215 may be stored and maintained in the database 115 using one or more files in various formats, such as BMP, TIFF, JPEG, GIF, and PNG, among others. The profile manager 125 may also store and maintain metadata for the images 215 identifying or including the annotation for the images 215 and the user identifier for the user 205.

In addition, at least a portion of the images 215 may be obtained from human subjects outside the users 205 of the application 150. In some embodiments, the profile manager 125 may maintain another subset of images 215 in the database 115 that are not associated with any users 205 of the application 150. For example, such images 215 may be obtained by the profile manager 125 from a corpus or facial expression database, with labels of facial expressions together with their respective intensities. In some embodiments, the labels of the images 215 identifying the type of facial expressions and relative intensities may be manually annotated by a human (e.g., a clinician) examining the images 215. In some embodiments, the labels of the images 215 identifying the type of facial expressions and relative intensities may be generated using automatic facial expression recognition algorithm.

To start a session trial, the application 150 on the client 110 may provide, send, or otherwise transmit at least one request 220 for the session trial for the user 205 of the application 150 to the session management service 105. The session trial may correspond to an instance of a task (e.g., Implicit Association Task (IAT)) that the user 205 is to carry out and be evaluated on. The request 220 may include or identify the user 205 to which the session trial is to be provided. In some embodiments, the application 150 may send the request 220 in response to detecting a corresponding interaction with the interface 155 of the application 155. In some embodiments, the application 150 may send the request 220 independent of interaction by the user 205 with the interface 155, for example, in accordance with a schedule for addressing the condition of the user 205.

Upon receipt of the request 220 from the client 110, the profile manager 125 may select or identify the user profile 210 corresponding to the user 205 from the database 115. With the identification, the profile manager 125 may parse the user profile 210 to identify the included information, such as the user identifier and the condition to be addressed (e.g., chronic pain), among others. The profile manager 125 may also identify the information on previous session trials and the performance metric for the user 205, among others. Using the user profile 210, the profile manager 125 may identify a subset set of images 215 associated with the user 205 in the database 115. For instance, the profile manager 125 may search the database 115 for images 215 associated with the user 205 by finding images 215 labeled using metadata with the user identifier for the user 205.

For each session trial to be provided to the user 205, the image selector 130 executing on the session management service 105 may identify or select at least one self image 225A (sometimes generally referred herein as a first image 225A). The image selector 130 may select the self image 225A from the subset of images 215 identified as from the user 205 in the database 115. The self image 225A may be associated with the user 205 and may be, for example, an image of a facial expression of the user 205. The selection of the self image 225A may be at random or in accordance with a selection policy. The selection policy may define a rule set (e.g., probability, decision tree, or a sequence) with which to select the self images 225A and may be dependent on a previous response to a prior session trial from the user 205. For example, the rules may specify that a self image 225A of a facial expression associated with pain (e.g., in angst) at various intensities is to be selected at 50-60% of the time and a self image 225A of a facial expression disassociated with pain (e.g., relaxed) at various intensities is to be selected 40-50% of the time, depending on the performance of the user 205 in the session trials.

In addition, the image selector 130 may identify or select at least one other image 225B (sometimes generally referred herein as a second image 225B). The image selector 130 may select the other image 225B from images 215 besides the user 205 in the database 115. The other image 225B may be selected from images 215 of other users 205 of the application 150. The other image 225B may be selected from images 215 of human subjects outside the users 205 of the application 150 (e.g., from the facial expression corpus). In some embodiments, the other image 225B may have the same type of facial expression as the selected self image 225A. In some embodiments, the other image 225B may have a different type of facial expression from the type of facial expression in the self image 225A. The intensity level for the facial expression in the other image 225B may be the same as or may differ from the intensity level for the facial expression in the self image 225A.

Furthermore, the image selector 130 may identify or select at least one stimulus image 225C (hereinafter generally referred to as a third image 225C). The image selector 130 may select the stimulus image 225C from images 215 besides the user 205 in the database 115. The stimulus image 225C may be selected from images 215 of other users 205 of the application 150. The stimulus image 225C may be selected from images 215 of human subjects outside the users 205 of the application 150 (e.g., from the facial expression corpus). The stimulus image 225C may be selected in accordance with the selection policy. The stimulus image 225C may be associated with or may correspond to the condition of the user 205 as identified in the user profile 210. The stimulus image 225C may have an associative type or a non-associative (or dissociated or neutral) type of correspondence with the condition of the user 205. The stimulus image 225C may have the associative type, when the facial expression in the stimulus image 225C is associated with the condition. For example, for the condition of pain, the stimulus image 225C with the associative type may have facial expression of grimacing or angst. In contrast, the stimulus image 225C may have the non-associative type, when the facial expression in the stimulus image 225C is not associated with the condition. For instance, the stimulus image 225C with the non-associative type may have a facial expression of happy or relaxed.

In some embodiments, the image selector 130 may select the stimulus image 225C from the images 215 of other subjects based on previous session trials in accordance with the selection policy. In some embodiments, the selection of the stimulus image 225C based on an intensity level or a facial expression type of the previously selected stimulus image 225C and the performance of the user 205 as identified in the user profile 210. For example, when the performance metric of the user 205 is low indicating multiple incorrect responses, the image selector 130 may select the stimulus image 225C of a higher intensity level to further distinguish and increase the likelihood of a correct response. Conversely, when the performance metric of the user 205 is higher indicating multiple correct responses, the image selector 130 may select the stimulus image 225C of a lower intensity level to further test the user 205. In some embodiments, the selection of the stimulus image 225C in terms of intensity level may be at random or in accordance with the selection policy. For example, the rules may specify that a self image 225C of a facial expression associated with pain (e.g., in angst) at a first range of intensities is to be selected at 50-60% of the time and a stimulus image 225C of a facial expression associated with pain at a second range of intensities is to be selected 40-50% of the time. The selection may be dependent on the performance metric of the user 205 from previous session trials.

In some embodiments, the image selector 130 may select stimulus image 225C based on the task in accordance with the selection policy. The selection policy may define a rule set (probability, decision tree, or a sequence) with which to select the stimulus image 225C. The selection of the stimulus image 225C as defined by the selection policy may be dependent on a type of task to be performed by the user 205 and the facial expression type in the self image 225A or the other image 225B, among other factors. The task may include, for example, an associative task (e.g., association of the stimulus image 225C with the self image 225A) or a dissociative task (e.g., the association of the stimulus image 225C with the other image 225B), among others, with respect to the condition. For example, the rules may specify that the associative task is to be performed 40-60% and the dissociative task is to be performed 60-40% across the trials, depending on the performance of the user 205 in the session trials.

In selecting an image, the image selector 130 may identify or determine the task to be performed by the user 205 using the selection policy. In conjunction, the image selector 130 may identify the facial expression type for the self image 225A or the other image 225B. With the determination of the task and the facial expression type, the image selector 130 may identify candidate images 215 from the database 115. The candidate images 215 may correspond to images 215 besides the user 205. When the associative task is to be undertaken, the image selector 130 may select one of the candidate images 215 with a facial expression type not associated with the condition as the stimulus image 225C. The selected stimulus image 225C may have the non-associative type of correspondence with the condition. For example, the image selector 130 may select the image 215 with a smiling face that is not associated with the condition of chronic pain as the stimulus image 225C. On the other hand, when the dissociative task is to be undertaken, the image selector 130 may select one of the candidate images 215 with a facial expression type associated with the condition as the stimulus image 225C. The selected stimulus image 225C may have the associative type of correspondence with the condition. The intensity level for the facial expression in the stimulus image 225C may be the same as or may differ from the intensity level for the facial expression in the stimulus image 225A.

In some embodiments, the image selector 130 may select the stimulus image 225C using a threshold for the intensity level at which the stimulus images 225C is to be selected for the session trial. The threshold may be determined or defined by the selection policy. The image selector 130 may calculate, generate, or otherwise determine the threshold as a function of the expression type and intensity level of the selected self image 225A, the expression type and the intensity level of the selected other image 225B, and the performance metric of the user 205 in previous trial sessions, among others. The function may be defined by the selection policy. For example, the function may specify a higher threshold when the performance metric of the user 205 is relatively low and the facial expression of one of the selected images 225A and 225B is not associated with the condition. Conversely, the function may specify a lower threshold when the performance metric of the user 205 is relatively high and the facial expression of one of the selected images 225A and 225B is associated with the condition.

To select an image, the image selector 130 may identify images 215 in the database 115 besides images 215 of the user 205. From the identified images 215, the image selector 130 may identify the facial expression type and the intensity level of each image 215. Upon identification, the image selector 130 may compare the intensity level of each image 215 with the threshold. If the intensity level satisfies (e.g., is greater than or equal to) the threshold, the image selector 130 may include the image 215 in a candidate set for the stimulus image 225C. Otherwise, if the intensity level does not satisfy (e.g., is less than) the threshold, the image selector 130 may exclude the image 215 from the candidate set for the stimulus image 225C. From the remaining images in the candidate set, the image selector 130 may select one image 215 to use as the stimulus image 225C in accordance with the selection policy. The selection policy may define whether to select an image 215 with the same or a different facial expression type as the self image 225A to use as the stimulus image 225C.

With the selection of the self image 225A, the other image 225B, and the stimulus image 225C (e.g., collectively, the images 225), the package generator 135 executing on the session management service 105 may generate or determine trial parameters 230. The trial parameter 230 (sometimes herein referred to as display parameters, presentation parameters, or session parameters) may define the presentation of the images 225 within the user interface 155 of the application 150. The trial parameters 230 may specify, identify, or otherwise define any or all of the following: start times at which to initiate display of the self image 225A, the other image 225B, and the stimulus image 225C, respectively; end times at which to cease displaying of the self image 225A, the other image 225B, and the stimulus image 225C, respectively; lengths (or durations) of presentation of the self image 225A, the other image 225B, and the stimulus image 225C, respectively; locations (e.g., pixel coordinates) of the self image 225A, the other image 225B, and the stimulus image 225C within the user interface 155; sizes (or dimensions) of the self image 225A, the other image 225B, and the stimulus image 225C, respectively, on the user interface 155; a relative location of the stimulus image 225C with respect to the self image 225A and the other image 225B; and a relative size of the stimulus image 225C with respect to the self image 225A and the other image 225B, among others.

The package generator 135 may use the user profile 210 to determine or generate the trial parameters 230. The setting of the trial parameters 230 based on the user profile 210 may be to adjust (e.g., increase or decrease) the likelihood of correct response or selection by the user 205. In some embodiments, the package generator 135 may assign, set, or otherwise determine relative start times for the self image 225A, the other image 225B, and the stimulus image 225C based on the performance metric. For example, if the performance metric is relatively low (e.g., compared to a baseline), the package generator 135 may determine to further spread the start times of the self image 225A relative to the other image 225B and the stimulus image 225C to provide the user 205 a longer time for reference to increase the likelihood of correct response. In some embodiments, the package generator 135 may assign, set, or otherwise determine a length of the presentation of the stimulus image 225C based on the performance metric of the user 205 from previous session trials. For instance, the package generator 135 may set the length of the display to be higher to increase the likelihood of correct response, when the performance metric of the user 205 is relatively low (e.g., compared to a baseline).

Continuing on, in some embodiments, the package generator 135 may assign, set, or otherwise determine a location of the presentation of the stimulus image 225C relative to the self image 225A or the other image 225B based on the performance metric of the user 205 from previous session trials. For example, the package generator 135 may set the location of the stimulus image 225C to be closer to the self image 225A, when the performance metric of the user 205 is relatively low (e.g., compared to a baseline) and the task to be performed is associative. The setting of the stimulus image 225C closer to the self image 225A than the other image 225B may increase the likelihood of the user 205 to make the correct selection. Conversely, the setting of the stimulus image 225C further from the self image 225A than the other image 225B to increase the difficulty of correctly performing the task. In some embodiments, the package generator 135 may assign, set, or otherwise determine a size of the presentation of the self image 225A, the other image 225B, and the stimulus image 225C relative to one another based on the performance metric of the user 205 from previous session trials. For instance, the package generator 135 may enlarge the size of the self image 225A relative to the size of the other image 225B when the performance metric of the user 205 is relatively low (e.g., compared to a baseline) and the task to be performed is associative.

In some embodiments, the package generator 135 may identify or determine a correct response (or selection) for the session trial including the set of images 225. The determination of the correct response may be in accordance with the task as defined by the selection policy used to select the images 225, such as the stimulus image 225C. When the task to be performed is the associative task, the package generator 135 may determine the association of the stimulus image 225C with the self image 225A is the correct response. The package generator 135 may further determine the association of the stimulus image 225C with the other image 225B is the correct response. Otherwise, when the task to be performed is the dissociative task, the package generator 135 may determine the association of the stimulus image 225C with the other image 225B is the incorrect response. Furthermore, the package generator 135 may determine the association of the stimulus image 225C with the self image 225A is the incorrect response.

Upon determination, the package generator 135 may output, create, or otherwise generate at least one session package 235. The session package 235 may identify, contain, or otherwise include information to run the session trial for the user 205 through the application 150 on the client 110. The session package 235 may identify or include the self image 225A, the other image 225B, the stimulus image 225C, and the trial parameters 230. In some embodiments, the session package 235 may include identifiers (e.g., uniform resource locators (URLs)) referencing the self image 225A, the other image 225B, and the stimulus image 225C, respectively. In some embodiments, the session package 235 may also identify or include an identification of the task, the correct response, or incorrect response, among others. In some embodiments, the session package 235 may correspond to one or more files (e.g., image and configuration files) to be sent to the application 150. In some embodiments, the session package 235 may correspond to the payload of one or more data packets sent to the application 150 on the client 110. With the generation, the package generator 135 may transmit, send, or otherwise provide the session package 235 to the application 150 on the client 110.

The application 150 executing on the client 110 may retrieve, identify, or otherwise receive the session package 235 from the session management service 105. Upon receipt, the application 150 may process and load the session package 235 for presentation on the user interface 155. The application 150 may parse the session package 235 to extract or identify the self image 225A, the other image 225B, the stimulus image 225C, and the trial parameters 230. Upon identification, the application 150 may initiate presentation of the self image 225A, the other image 225B, and the stimulus image 225C in accordance with the trial parameters. For instance, the application 150 may start rendering of the self image 225A, the other image 225B, and the stimulus image 225C in the user interface 155 at the specified respective start times for the defined durations. The application 150 may set the locations and sizes of the renderings of the self image 225A, the other image 225B, and the stimulus image 225C as defined by the trial parameters 230. In addition, the application 150 may render, present, or otherwise provide at least one user interface element (e.g., a scroll bar, a command button, check box, radio button, or a message prompt) in the user interface 155 for associating the stimulus image 225C with one of the self image 225A or the other image 225B. The application 150 may continue rendering the self image 225A, the other image 225B, and the stimulus image 225C until the respective end times as defined by the trial parameters 230.

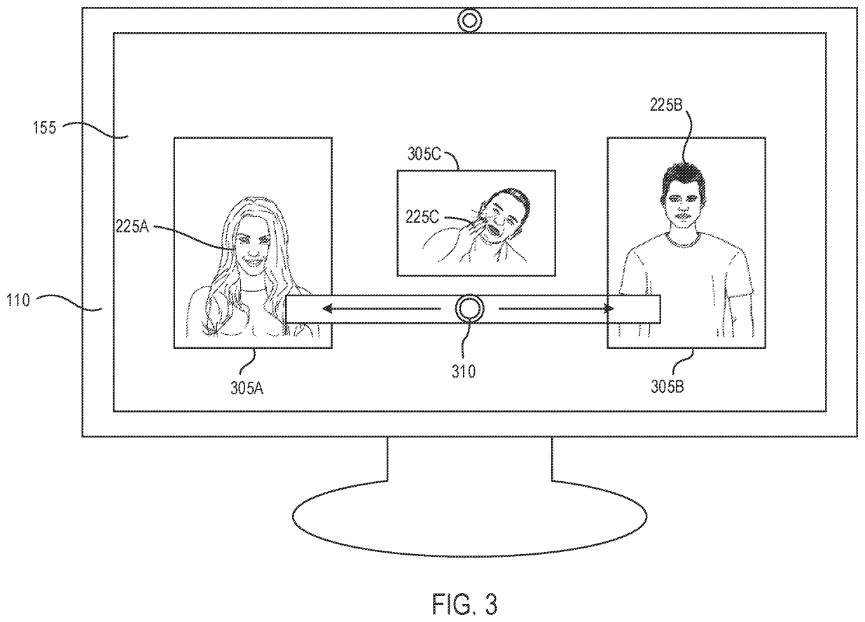

Referring now to FIG. 3, depicted is a block diagram of an example of the interface 155 for a session presented to subjects. The depicted example may be an example presentation of the user interface 155 by the application 150 running on the client 110 using the session package 235. In the depicted example, the user interface 155 presented in the display of the client 110 may include the self image 225A generally along the left within an image element 305A and the other image 225B generally along the right within an image element 305B. The self image 225A and the other image 225B may both have facial expressions (e.g., smiling and composed, respectively) not related to the condition (e.g., chronic pain) of the user 205. The self image 225A and the other image 225B may have similar dimensions.

Between the two images, the user interface 155 may include the stimulus image 225C within an image element 305C generally toward the middle. The stimulus image 225C may have size relatively smaller than the sizes of the self image 225A and the other image 225B. The stimulus image 225C may have a facial expression (e.g., grimacing in pain) related to the condition. In addition, the user interface 1155 may include at least one user interface element 310. The user 205 may use the user interface element 310 to associate the stimulus image 225C with the self image 225A or the other image 225B. Since the type of task to be performed by the user 205 may be a dissociative task, the user 205 may be expected to use the user interface element 310 to associate the stimulus image 225C with the other image 225B. The user may do so by sliding the button in the middle of the user interface element 310 toward the right.

As detailed below, at each iteration of the session trial, the session package 235 may be generated to account for user response data. Upon receipt of the session package 235, the application 150 running on the client 110 may be able to present the images 225 that are more targeted to the particular characteristics of the user 205 (e.g., presentation duration of the images 225, resizing and repositioning of the images 225 relative to one another, and with adjusted intensity levels for facial expressions in the stimulus image 225C), thereby making the overall session trial more relevant to the user 205. This may have the effect of improving the quality of human-computer interactions (HCI) between the user 205 and the application 150 through the user interface 155. In conjunction with the improvement in HCI, the specifications of the session package 235 may allow the user 205 to more accurately respond, thereby training the user 205 to perform the session trials properly with less iterations and reducing consumption of computing resources on the client 110 (e.g., processor, memory, and power).

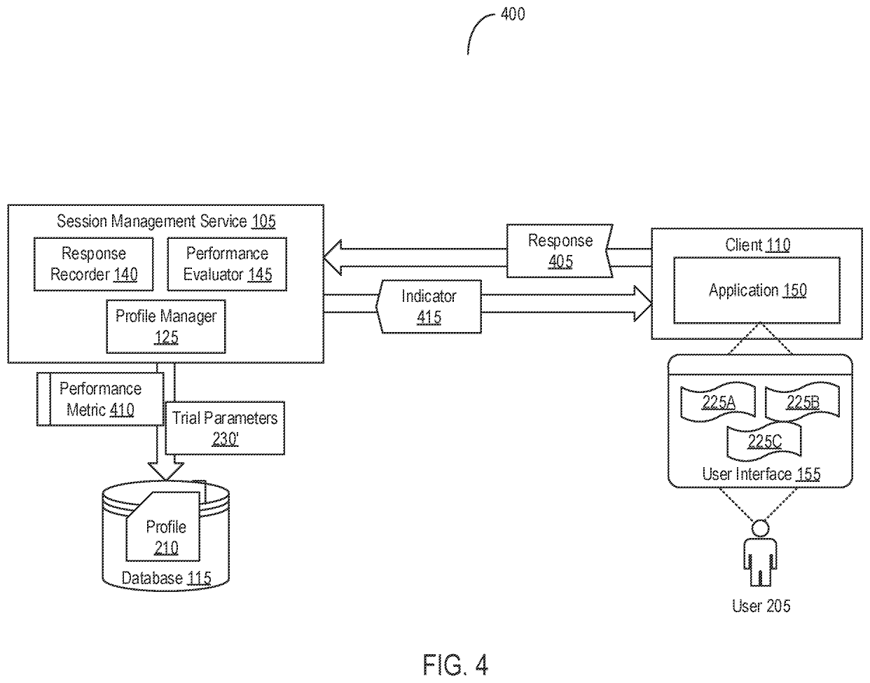

Referring now to FIG. 4, among others, depicted is a block diagram of a process 400 of recording subject responses in the system 100 for a performing session. The process 400 may include or correspond to operations in the system 100 to receive responses for session trials and determine performance metrics in carrying out the task for the session trial. Under the process 400, the application 150 running on the client 110 may monitor for at least one interaction from the user 205 with the user interface 155. The interaction may indicate or identify an association of the stimulus image 225C with one of the self image 225A or the other image 225B for the session trial. The application 150 may use an event listener or a handler of one or more of the user interface elements (e.g., the user interface element 310) of the user interface 155 to monitor and handle the interaction from the user 205.

With the detection of the interaction, the application 150 may output, produce, or otherwise generate at least one response 405. The response 405 may identify the association of the stimulus image 225C with one of the self image 225A or the other image 225B from the user interaction with the user interface 155. In conjunction, the application 150 may measure, calculate, or otherwise determine the time elapsed between the presentation of the stimulus image 225C and the detection of the interaction from the user 205. The application 150 may use the timer to determine the elapsed time between the initial presentation of the stimulus image 225C and the interaction. The response 405 may also identify or include the user identifier of the user 205. Upon generation, the application 155 may provide, transmit, or otherwise send the response 405 to the session management service 105.

The response recorder 140 executing on the session management service 105 may in turn retrieve, identify, or otherwise receive the response 405 from the client 110. Upon receipt, the response recorder 140 may parse the response 405 to extract or identify the user interaction as associating the stimulus image 225C with one of the self image 225A or the other image 225B. In addition, the response recorder 140 may identify the elapsed time between the presentation of the stimulus image 225C and the detection of the interaction from the user 205. With the identification, the response recorder 140 may store and maintain the response 405 onto the database 115, such as on an interaction log for the user 205. The response recorder 140 may store an association between the response 405 and the user profile 210 using the user identified as the user 205. In some embodiments, the response recorder 140 may store and maintain identifications of the images 225 (e.g., the self image 225A, the other image 225B, and the stimulus image 225C) of the session trial in the database 115. The response recorder 140 may store an association between the identifications of one or more of the images 225 with the user profile 210.

The performance evaluator 145 executing on the session management service 105 may calculate, generate, or otherwise determine at least one performance metric 410 of the user 205 for the session trial. The determination of the performance metric 410 (sometimes herein referred to as a score or metric) may be based on the association identified in the response 405 and the type (e.g., associative or non-associative) of correspondence of the stimulus image 225C with the condition of the user 205. The performance metric 410 may be a value (e.g., numeric value) identifying or corresponding to the ability of the user 205 to correctly perform the task for the session trial as defined by the session package 235. The performance metric 410 may be assigned or set to one value when association in the response 405 is correct and another value when the association in the response 405 is incorrect. The performance metric 410 may be used to select images 225 and determine trial parameters 230 for subsequent session trials.

For the associative type of correspondence, when the association is between the stimulus image 225C with the self image 225A, the performance evaluator 145 may set the performance metric 410 to indicate the response 405 as an incorrect response. For instance, the performance evaluator 145 may assign a value (e.g., “−10”) to the performance metric 410 to indicate the incorrect response. Conversely, when the association is between the stimulus image 225C with the other image 225B, the performance evaluator 145 may set the performance metric 410 to indicate the correct response. For example, the performance evaluator 145 may assign a value (e.g., “10”) to the performance metric 410 to indicate the correct response.

For the non-associative type of correspondence, when the association is between the stimulus image 225C with the self image 225A, the performance evaluator 145 may set the performance metric 410 to indicate the response 405 as an correct response. For instance, the performance evaluator 145 may assign a value (e.g., “10”) to the performance metric 410 to indicate the correct response. Conversely, when the association is between the stimulus image 225C with the other image 225B, the performance evaluator 145 may set the performance metric 410 to indicate an incorrect response. For example, the performance evaluator 145 may assign a value (e.g., “−10”) to the performance metric 410 to indicate the incorrect response.

In some embodiments, the performance evaluator 145 may determine the performance metric 410 as a function of whether the response 405 identifies the correct selection, the images 225 provided, the intensity levels for the stimulus image 225C (or other images 225), and the elapsed time between the presentation of the stimulus image 225C and the interaction by the user 205, among others. For instance, the function may define an adjustment amount for the value initially assigned based on the correctness of the response 405 to account for the response time of the user 205 as measured by the elapsed time between presentation of the stimulus image 225C and the interaction. The function may specify a threshold time for the stimulus image 225C at which to apply the adjustment amount. In general, the performance metric 410 for a correct response with a relative shorter response time may be greater than the performance metric 410 for an incorrect response or a correct response with a relatively longer response time. The function may also specify adjustment amounts depending on the intensity level of the stimulus image 225C provided to the user 205 in the session trial.

To determine whether to apply the adjustment, in some embodiments, the performance evaluator 145 may compare the elapsed time with the threshold time for the stimulus image 225C. If the elapsed time is greater than the threshold time, the performance evaluator 145 may apply the adjustment amount to the initially assigned value for the performance metric 410 in accordance with the function. In contrast, if the elapsed time is less than or equal to the threshold time, the performance evaluator 145 may maintain the initially assigned value for the performance metric 410. In some embodiments, the performance evaluator 145 may identify the intensity level of the stimulus image 225C. Based on the intensity level, the performance evaluator 145 may modify the value of the performance metric 410 in accordance with the function. With the determination, the performance evaluator 145 may store and maintain the performance metric 410 in the database 115.

In some embodiments, the performance evaluator 145 may transmit, send, or otherwise provide at least one indicator 415 to the application 150. The indicator 415 may identify whether the association in the response 405 is the correct selection or the incorrect selection for the user 205 of the application 150 on the client 110. The performance evaluator 145 may generate the indicator 415 based on the determination of the performance metric 410. When the determination of the performance metric 410 is to indicate the correct response, the indicator 415 may identify the association as the correct response. Otherwise, when the determination of the performance metric 410 is to indicate the incorrect response, the indicator 415 may identify the association as the incorrect response. Upon generation, the performance evaluator 145 may send the indicator 415 to the application 150 on the client 110.

The application 150 may in turn retrieve, identify, or receive the indicator 415 from the session management system 110. Upon receipt, the application 150 may parse the indicator 415 to extract or identify the identification of the association as the correct response or the incorrect response. With the identification, the application 150 may render, display, or otherwise present the indicator 415 identifying the association as the correct or incorrect response to the user 205. For example, the application 150 may display a user interface element (e.g., a text box, an image, or prompt) indicating the association as the correct or incorrect response, or play a video or audio file indicating the association, as identified in the indicator 415, among others. In some embodiments, the application 150 may present an indication of the association by the user 205 as incorrect or correct. For instance, the application 150 may determine whether the user interaction with the user interface 155 is correct or incorrect as defined by the session package 235. Based on the determination, the application 150 may generate and present the indication identifying the association as correct or incorrect.

With the determination, the profile manager 125 may modify or update the user profile 210 using the performance metric 410. The profile manager 125 may store and maintain the performance metric 410 in the database 115 using one or more data structures, such as an array, a matrix, a table, a tree, a heap, a linked list, a hash, or a chain, among others. The profile manager 125 may store and maintain an association between the user profile 210 for the user 205 and the performance metric 410. In some embodiments, the profile manager 125 may adjust, modify, or otherwise update the performance metric 410 identified in the profile 210. For instance, the profile manager 125 may update the performance metric 410 as a function of previously determined performance metrics 410. The function may be, for example, a moving average (e.g., unweighted, cumulative, or exponentially weighted). With the update, the profile manager 125 may store the new value for the performance metric 410 in the user profile 210.

In addition, the profile manager 125 may determine or generate new trial parameters 230′ based on the performance metric 410. In some embodiments, the profile manager 125 may update the trial parameters 230 to generate the new trial parameters 230′ based on performance metrics 410 determined across multiple session trials for the user 205. The updated, new trial parameters 230′ may adjust (e.g., increase or decrease) the likelihood of correct response or selection by the user 205. To generate, the profile manager 125 may determine an aggregate value (e.g., weighted average, slope, or sum) for the performance metrics 410 over the previous set number of session trials. The aggregate value may correspond to the trend in performance of the user 205 in performing the session trials.

With the determination, the profile manager 125 may compare the aggregate value with a set of ranges. The set of ranges may identify ranges of values for the aggregate value of the performance metrics 410 at which to adjust the trial parameters 230. The set of ranges may include or identify any or all of the following: a first range at which to adjust to increase likelihood of correct selection (and by extension make the task easier); a second range at which to adjust to decrease likelihood of correct selection (and by extension make the task more difficult); or a third range at which to maintain the trial parameters 230. When the aggregate value is within the first range, the profile manager 125 may generate the new trial parameters 230′ to increase the likelihood of correct response and by extension make the task for the next session trial easier. For instance, the profile manager 125 may update the trial parameters 230 to increase the length of the presentation of the next selected stimulus image 225C, set the location the stimulus image 225C closer to the correct image 225 (e.g., self image 225A or the other image 225B), or determine the relative sizes of the image 225 (e.g., increase the stimulus image 225C), among others.

Continuing on, when the aggregate value is within the second range, the profile manager 125 may generate the new trial parameters 230′ to decrease the likelihood of correct response and by extension make the task for the next session trial more difficult for the user 205. For example, the profile manager 125 may update the trial parameters 230 to decrease the length of the presentation of the next selected stimulus image 225C, set the location the stimulus image 225C closer to the incorrect image 225 (e.g., self image 225A or the other image 225B), or determine the relative sizes of the image 225 (e.g., decrease the stimulus image 225C), among others. When the aggregate value is within the third range, the profile manager 125 may use the current trial parameters 230 for the next session trial. Upon determination, the profile manager 125 may store and maintain the new trial parameters 230′ for the next session trial in the user profile 210. The processes 200 and 400 may be repeated any number of times to train the user 205 to disassociate stimulus images 225C related to the condition (e.g., chronic pain) away from the user 205 or to associate stimulus images 225C not related to the condition with the user 205. With each iteration, the responses 405 from the user 205 in performing the task as defined by the session trials may be obtained and the trial parameters 230 may be adaptively modified using the performance metrics 410.

In this manner, the session management service 105 together with the application 150 may enable the user 205 to be provided with the images 225 as part of the session package 235 to carry out the tasks (e.g., Implicit Association Task (IAT)) anywhere, independent of the locations of the centralized session management service 105 as well as any laboratory or clinic. The user 205 may also easily access and view the stimuli (e.g., the self image 225A, the other image 225B, and the stimulus image 225C) for performing the tasks as define by the session trials. The ability to access the application 150 to carry out the tasks anywhere may improve the overall utility of the client 110 (or other computing device) in providing the session trial and digital therapeutics to such users 205.

Furthermore, the use of the rule set of the selection policy together with past responses may select images 225 for session trials in a regular manner that are pertinent to the condition of the user 205. By incorporating responses 405 and determining performance metrics 410, the session management service 105 may update the trial parameters 230 to dynamically configure and modulate the presentation of the images 225 (e.g., by duration, size, and positioning) through the user interface 155 in an objective fashion. In addition, the session package 235 generated by the session management service 105 may result in information being displayed on the user interface 155 that may be more readily relevant and comprehensible to the user 205 to induce the user 205 to increase the probability of making the correct selection. As a consequence, the session package 235 for the session trial may thus increase usefulness of responses 405 obtained from the user 205 in response to presenting the images 225.

The updating of the trial parameters 230 and the adaptive selection of the images 225 may also reduce and eliminate instances of multiple repeated trials with non-useful results, relative to approaches that do not rely on such iterative processes. With the reduction or elimination of repeated, fruitless session trials, the processes 200 and 400 may decrease or save computer resources (e.g., the processor, memory, and power) and network bandwidth used by the session management service 105, the client 110, and the overall system 100 that would otherwise be incurred thereby increasing the efficiency of these devices. Furthermore, the session package 235 together with the user interface 155 may reduce the number of interactions to be taken by the user 205 to accomplish a particular task, thus decreasing the amount of computing resources on the client 110 and increasing the quality of human-computer interaction (HCI) between the user 205 and the overall system 100.

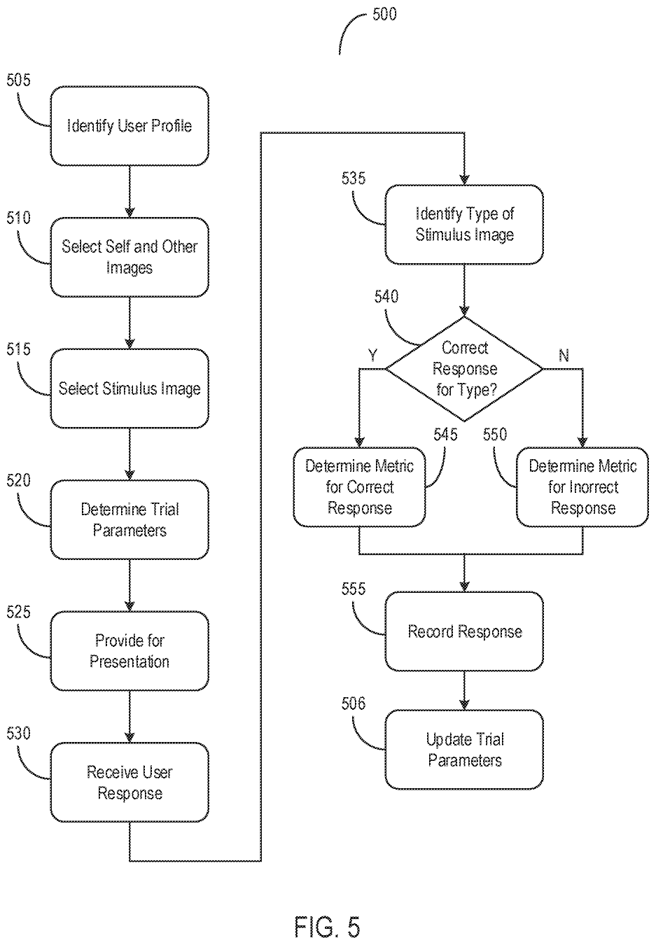

Referring now to FIG. 5, among others, depicted is a flow diagram of a method 500 of performing sessions for users to acquire response data. The method 500 may be implemented or performed using any of the components detailed herein, such as the session management service 105 or the system 800. Under the method 500, a computing system may identify a user profile (505). The computing system may select self and other images (510). The computing system may select a stimulus image (515). The computing system may determine trial parameters (520). The computing system may provide the session trial for presentation (525). The computing system may receive a user response (530). The computing system may identify a type of the stimulus image (535). Based on the type, the computing system may determine whether a correct response has been given (540). If the response is correct, the computing system may determine a metric for the correct response (545). Otherwise, if the response is incorrect, the computing system may determine the metric for incorrect response (550). The computing system may record the user response (555). The computing system may update the trial parameters (560).

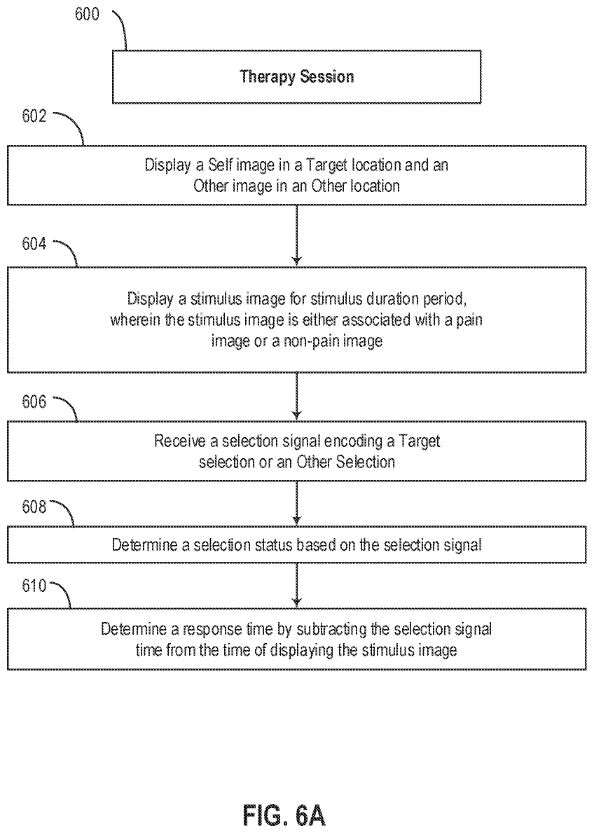

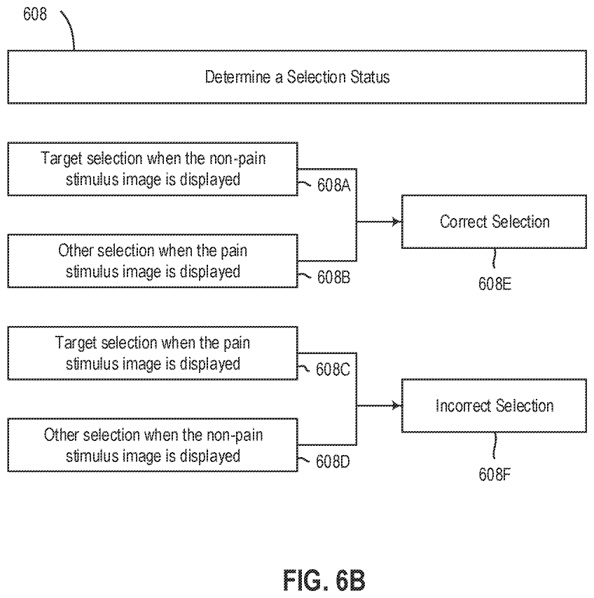

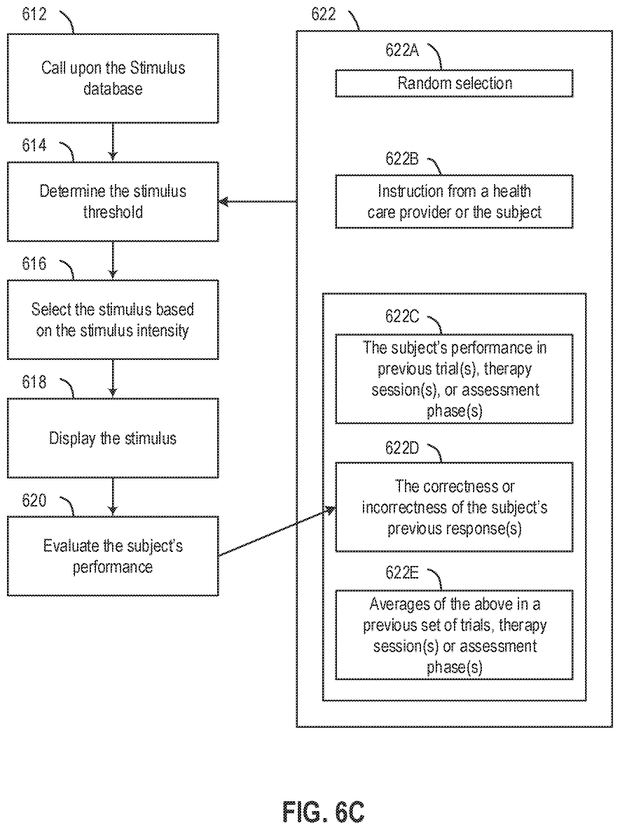

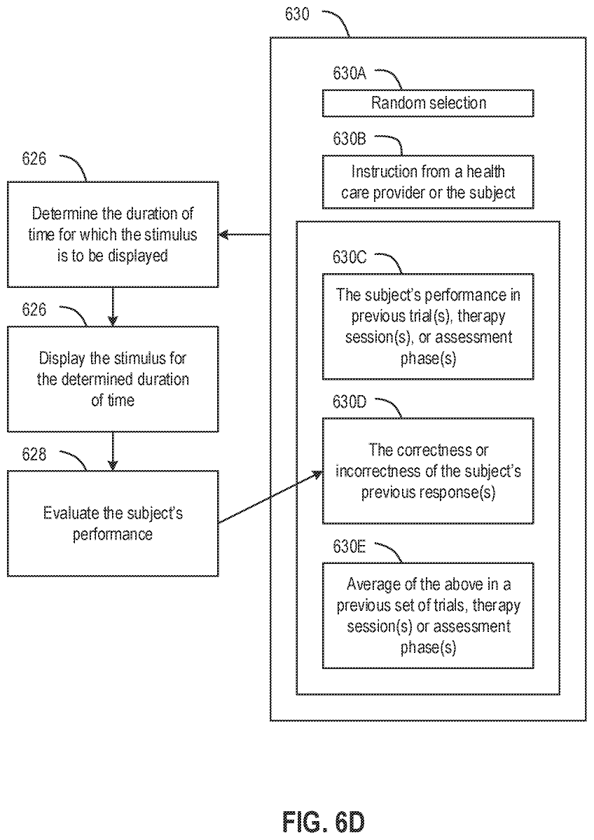

Referring now to FIGS. 6A and 6B, among others, depicted is a flow diagram of a method 600 of running trial sessions and a flow diagram of an embodiment of selection status determination respectively. In some embodiments, the method is carried out by one or more programs of the subject computer system described herein. In some embodiments, the method is for the treatment of a chronic pain disorder, which may include arthritis, migraine, fibromyalgia, back pain, Lyme disease, endometriosis, repetitive stress injuries, irritable bowel syndrome, inflammatory bowel disease, and cancer pain.

In some embodiments, the method comprises conducting a therapy session 600. The conducted therapy session comprises a predetermined number of trials, each trial comprising displaying 602 a self image in a Target location and a other image in another location; displaying 604 a stimulus image for a predetermined amount of time, the stimulus image either associated with pain or non-pain; receiving 606 a selection signal encoding a Target selection or another selection; determining 608 a selection status based on the selection signal, wherein the selection status comprises a correct or an incorrect selection, wherein the correct selection 608E comprises (a) the Target selection when the non-pain stimulus is displayed 608A or (b) the other selection when the pain stimulus is displayed 608B, and the incorrect selection 608F comprises (a) the Target selection when the pain stimulus is displayed 608C or (b) the other selection when the non-pain stimulus is displayed 608D; and determining 610 a response time equal to the time of receiving the selection signal minus the time of displaying the stimulus image.

In other words, the subject engages in repeated trials in which the subject may associate a non-pain stimulus with Self, and pain-related stimulus with other, as accurately and as quickly as possible. The self image may be a word or pictorial image that is associated with the subject, for example, the self image may be a pictorial image of the subject or the name of the subject. By engaging in repeated trials in which the subject associates the self with non-pain related stimuli, and the other with pain-related stimuli, the subject may be trained to focus pain-related ideas away from the self and develop a conceptualization of the self independent of, or less over-identified with, pain. Thus, the therapy is aimed at reducing the subject's maladaptive self-processing, particularly the subject's self-pain enmeshment.

The user interface 155 may include a self image 225A, an image element 305A, an other image 225B, and an image element 305B. The self image 225A is located in or at image element 305A, and the other image 225B is located in or at image element 305B. The self image 225A is associated with the subject, while the other image 225B is associated with a person other than the subject. In some embodiments, the subject image 302 is a depiction, likeness or image of the subject. In those embodiments, the other image 225B is a depiction, likeness or image of an individual other than the subject. The self image 225A and/or the other image 225B may comprise words, images, video, audio, haptic and/or olfactory elements, either individually or in combination. For instance, in some embodiments, the self image 225A is the subject's name, while the other image 225B is a different name. In some embodiments, the other image is any image other than that of the subject.

In some embodiments, the systems and methods for digitally treating chronic pain comprise steps of receiving and storing images in the database. For instance, images are uploaded by the subject or by a medical professional, received from a network, or received via a peripheral device such as a camera. In some embodiments, displaying the self image 225A and the other image 225B comprises engineering images that are related to the subject and/or the other. In some embodiments, displaying the self image 225A and the other image 225B comprises retrieving the self image 225A and other image 225B from the image database 250. Retrieving said images may further comprise retrieving other images 25B according to an algorithm.

The image element 305A and the image element 305B are locations on the user interface 155 in which the self image 225A and the other image 225B are displayed, respectively, such that a selection signal may be encoded as a Target selection or an other selection. In an embodiment, the selection signal is a signal generated when the subject selects any point within the image element 305A or Other Location 308, thereby making a selection of area corresponding to the self image 225A or other image 225B. In other embodiments, the selection signal is generated when the subject drags the stimulus to the image element 305A or the image element 305B. In other embodiments, the selection signal is generated when the subject presses a first key associated with the self image 225A or a second key associated with the other image 225B. In other embodiments, eye tracking software may be utilized to send a selection signal when the subject makes a selection with their gaze.

In some embodiments, the image element 305A is located on the left portion of display user interface 155 and the image element 305B on the right portion. In other embodiments, the image element 305A and image element 305B are located on opposite top and bottom portions of display user interface 155. The image element 305A and the image element 305B may be different sizes, and may be located in other portions of display user interface 155, including the image element 305A and image element 305B not necessarily located on opposite portions of the interface. In some embodiments, it may be advantageous to alter the image element 305A and image element 305B during a therapy session in order to increase the likelihood that the subject consciously chooses the association between stimuli and self or other, and decrease effects of the subject becoming accustomed to the usual location of the Self and other images.

The user interface 155 may include a stimulus image 225C. The stimulus image 225C comprises words, images, video, audio, haptic and/or olfactory elements, either individually or in combination, related to pain or non-pain. For example, the stimulus image 225C depicted in FIG. 3 is a picture of a person grimacing in pain—this would be a pain-related stimulus. Other examples of pain-related stimuli include images depicting facial expressions of pain; persons holding different parts of their body and showing faces expressing pain; mutilated, burnt, hurt, or otherwise visibly damaged body parts; words such as ‘pain’, ‘agonizing’, ‘hurt’, ‘pounding’, ‘aching’, ‘headache,’ etc. . . . . On the other hand, examples of non-pain related images may include images of neutral or relaxed facial expressions; healthy body parts without visible damage; depictions of pain-free body postures; words such as ‘pain-free’, ‘relaxed’, ‘ease’, ‘comfort’, ‘whole’, ‘healthy’, ‘able’, and ‘functioning’, among others. In some embodiments, the user interface 155 may also include a stimulus image 225C. The stimulus image 225C may reside within an image location 305C on user interface 155. Each stimulus image 225C may be configured to induce positive and negative emotional responses in the subject, and therefore achieve limbic system (ACC, PFC, amygdala, insula, VTA and NAc) activation and activation in the brain's self-referential networks (PCC, MPFC, insula, DMN network). Although pain-related stimuli are described here, it is further contemplated that the treatments described herein may be applicable to other maladaptive self-processing behaviors, including other associations between the self and negative concepts such self-criticism.

In some embodiments, the database 115 comprises the set of stimuli for display. In some embodiments, the systems and methods comprise steps of receiving and storing stimuli in the database 115. For instance, stimuli may be uploaded to the system by the subject or by a medical professional, received from a network, or received via a peripheral device such as a camera. In some embodiments, an additional step of engineering stimuli may be performed. In some embodiments, displaying the stimulus comprises retrieving a stimulus from the database 255.

In some embodiments, stimuli are associated with data related to each stimulus's relevance to the subject. For example, it may be determined during an onboarding step that the subject associates a particular set of stimuli with their pain, or the subject may be prompted to identify or rank stimuli as related or unrelated to their pain experience. In some embodiments this determination may be made by a health care provider. In other embodiments, stimuli's relevance to the subject may be determined by the subject's performance in a training session, or by algorithms or models that predict the stimuli's relevance to the subject.

In some embodiments, each stimulus is associated with an intensity. In some embodiments, the intensity of a stimulus image is determined by the subject and/or a health care provider. For example, systems and methods may comprise a step of receiving a ranking of intensities of a predetermined set of stimuli images, or receiving an assignment of intensity level to the predetermined set of stimuli images. In some embodiments, the intensity of a stimulus image is determined with reference to an algorithm or model that indicates the predicted intensity of an image to the subject. The model may be a machine learning model (e.g., reinforcement learning model, k-nearest neighbor model, backpropagation model, q-learning model, genetic algorithm model, neural networks, supervised learning model, unsupervised learning model, etc. . . . ). Said model may be trained using contextual data such as data received from the subject, from a network, and/or uploaded data. In some embodiments, the set of stimuli are engineered with predetermined intensities.

Referring now to FIG. 6C, depicted is a flow diagram of an embodiment of stimulus threshold determination and stimulus selection. In some embodiments, displaying a stimulus may comprise selecting the stimulus from the database 115 based on a stimulus threshold. The process of selecting the stimulus may begin by calling upon the Stimulus database 612. The stimulus threshold may be defined as the intensity level of the stimulus to be displayed. The stimulus threshold may vary between trials, or may be constant throughout the therapy session. The determination 614 of the stimulus threshold may be based on one or more stimulus threshold factors 622. The stimulus threshold may be random 622A or it may be determined based on an instruction from a health care provider or the subject 622B, or on the subject's performance 622C in previous trial(s), therapy session(s), or assessment phase(s), such as the subject's response time(s) in previous trial(s), the correctness or incorrectness of the subject's previous response(s) 622D, or averages 622E of the foregoing in a previous set of trials, therapy session(s) or assessment phase(s). In one example, only stimuli surpassing a predetermined intensity level are displayed in step 616. Accordingly, the selected stimulus may be displayed 618 to the subject. In one example, the factors 622 may be a function of the evaluation of the subject's performance 620. In such an example, the stimuli threshold may be increased when the response time(s) of the subject is determined to be under a threshold and/or when the correct to incorrect response ratio of the subject is above a threshold, in other words, when the subject's performance is good.

In some embodiments, the therapy session comprises a predetermined number of trials, in which the number of pain stimuli and non-pain stimuli in the trials is determined by a stimuli ratio. For example, the stimulus ratio may be configured such that the non-pain stimulus is shown in at least 51% of trials (for example, to coerce the subject to more frequently match a non-pain stimulus to the Target). However, there is no specification that the non-pain stimulus be shown at least 51% of the time. Treatment may be accomplished by repeatedly prompting the subject to accurately and quickly pair non-pain stimuli with the self image 225A, or pain-stimuli with the other image 225B.

Referring now to FIG. 6D, depicted is a flow diagram of an embodiment of stimulus display period determination diagram. In each trial, the stimulus image 225C is displayed for a duration of time (the “stimulus display period”), which may range from 15 ms to 2 minutes. The stimulus display period may vary between trials, or may be constant through the therapy session. Thus, the stimulus delay period may be determined 624 based on one or more stimulus delay period factors 630. The duration of time the stimulus image 225C is displayed may be random 630A or it may be determined based on an instruction 630B from a health care provider or the subject, or on the subject's performance 630C in previous trial(s), therapy session(s), or assessment phase(s), such as the subject's response time(s) in previous trial(s), the correctness or incorrectness of the subject's previous response(s) 630D, or averages 630E of the foregoing in a previous set of trials, therapy session(s) or assessment phase(s). Once the stimulus display period has been determined, the stimulus may be displayed to the subject for said period 626. The stimulus display period factors 630 may be modified based on an evaluation of the subject's performance 628. For example, to adjust the difficulty of the trial, the duration of time of the display of a stimulus may be decreased when the response time(s) of the subject is determined to be under a threshold and/or when the correct to incorrect response ratio of the subject is above a threshold, in other words, when the subject's performance is good.

The subject may be prompted to associate the stimulus image 225C with the self image 225A or the other image 225B correctly and/or within the predetermined amount of time that the stimulus image 225C has been displayed. In some embodiments, a trial time may be longer than the predetermined amount of time in which the stimulus image 225C is displayed, and the subject is to associate the stimulus image 225C with the self image 225A or the other image 225B within the trial time. In some embodiments an incorrect association may result in displaying an error message, for example, a buzzing noise, pop-up window, or other animation configured to inform the subject that they have made an incorrect selection, and/or may or may not permit the subject to continue after an unsuccessful pairing. In other embodiments, no error message is displayed; in still other embodiments, an overall score or error rate for the therapy session is displayed at the end of a set of trials, or at the end of a therapy session.

In some embodiments, a trial further comprises the presentation of a blank screen preceding and/or following the user interface 155 screen. The blank screen may appear for a predetermined amount of time from 0 to 500 ms. The duration of the blank screen may be referred to as the inter-stimulus interval (ISI). In some embodiments, an indicator known as a fixation cross may be displayed in the location of the stimulus before the step of displaying the stimulus.

The system may include any suitable means for associating the stimulus with the image element 305A or image element 305B. For example, the subject may select the self image 225A and/or other image 306 by clicking (for example, with a mouse cursor), tapping (for example, with a touch screen and the subject's finger or stylus), or otherwise indicating a selection. In another embodiment, the subject may select the self image 225A or the other image 306 by swiping the screen (for example, dragging a finger across a touch screen). In such an embodiment, the subject may swipe in the direction of the self image 225A or other image 225B to select. In an embodiment, the stimulus image 225C may be selectable. In such an embodiment, the stimulus image 225C may have a passive state (for example, presented in a static manner on the user interface 155) and an active state (for example, movable by the subject). The stimulus image 225C may enter an active state when a subject presses the stimulus image 225C (for example, via a finger, mouse click, or other means). In an active state, the stimulus image 225C may become embossed, bold, glow, or otherwise change appearance. In an embodiment, in the active state, the stimulus image 225C may be moved by the subject. As a non-limiting example where the system includes a touch screen, the stimulus image 225C may be generated in a passive state and may be converted to an active state when a subject presses their finger on the stimulus image 225C, enabling the subject to “drag” or “swipe” the stimulus image 225C to either the image element 305A or image element 305B. The stimulus image 225C may return to the passive state upon the subject's removal of their finger from the stimulus image 225C and/or touch screen. In one embodiment, as shown in FIG. 3, the user interface 155 may include a selection tool 314 configured as an interactive slider. In such an embodiment, the subject may touch and drag the selection tool 314 to the desired location, for example, the image element 305A or image element 305B.

In alternate embodiments, the selection tool may comprise keys, buttons, a mouse, a track pad, or other means of allowing the subject to make a selection. For example, a first key may be associated with the self image 225A and a second key may be associated with the other image 225B, such that pressing the key comprises a selection. In another embodiment, the system includes a microphone that enables the subject to make vocal confirmations and selections. In such an embodiment, the subject may be able to answer the prompts by vocalizing their selection.

Upon receiving a selection signal, a selection status is determined in step 608. The selection status comprises a correct or an incorrect selection. The selection status may also comprise an error or non-responsive selection. A correct selection comprises (a) a non-pain stimulus is associated with the self image 225A, or (b) the pain-related stimulus is associated with the other image 225B, while an incorrect selection comprises (a) the pain-related stimulus is associated with the self image 225A, or (b) the non-pain stimulus is associated with the other image 225B. A response time (RT) is equal to the time at which the selection signal is received (or the predetermined stimulus display time if a selection signal is not received) minus the time the stimulus is presented (or the start of the trial, assuming the stimulus is presented at the same time after the start of each trial). As a non-limiting example, if the subject matched the stimulus image 225C to the image element 305A at 11:23:05 AM and the stimulus image 225C was presented at 11:23:04 AM, then the RT would be 1 second or 1000 milliseconds.

A digital assessment for chronic pain may comprise a predetermined number of trials after which a baseline maladaptive self-enmeshment (MSE) score may be determined. Each trial in the assessment phase may comprise the same elements as each trial in a therapy session as previously described. After the predetermined number of trials, the average RT of all incorrect responses is compared to the average RT of all correct responses, to determine the MSE score.

In one embodiment, the mean RT and standard deviation (SD) for all trials is calculated. In one embodiment, any trials where the RT is less than or greater than 2 SD beyond the mean RT are excluded from further calculations. However, there exist alternative embodiments, where the trials where the RT is less than or greater than 2 SD beyond the mean RT bear some weight or otherwise affect the non-excluded trials or final calculation in some suitable manner. There exist alternative embodiments where the median RT is calculated. Trials where the RT is less than or greater than 2 SD beyond the median RT may be excluded from further calculations. The system may also calculate the range, mode, or other data characteristics of the correct trials and/or incorrect trials.

In an embodiment, the assessment phase may include 100 to 1,000 trials. However, there exist alternate embodiments where the assessment phase may include less than 100 trials or more than 1,000 trials. In one embodiment, during the assessment phase an equal number of pain related stimuli and non-pain related stimuli are presented. Similarly, during the training phase, the system may generate an equal number of pain related stimuli and non-pain related stimuli. However, in another embodiment, a pain ratio may dictate the ratio of pain to non-pain stimuli that are presented during the training phase. In these various embodiments, the pain ratio may be weighed in the final calculation of RT.

In certain embodiments, the system 100 further includes a subject database preferably containing a plurality of subject profiles. In some embodiments, each subject profile contains subject information, such as, but not limited to, performance scores of the trials and therapy sessions described herein, pre and post therapy session assessments, and/or therapy session histories. Accordingly, the subject database may include at least the RT data, whether or not the user made the correct selection (selection status), and/or the number of trials that the user has performed. In certain embodiments, the subject profile further includes subject contact details, information concerning the subject's medical history, the subject's medical insurance details, etc. In some embodiments, the subject database also comprises information regarding psychiatric disorder treatment plans such as, but not limited to, the frequency of conducting therapy sessions describe herein, the absolute number of times that the therapy sessions are conducted, and/or any pharmaceuticals prescribed or other treatments (e.g., medication and other psychotherapies that target the brain regions and neural networks related to the psychiatric disorder being treated) administered concurrently with the treatments provided herein.

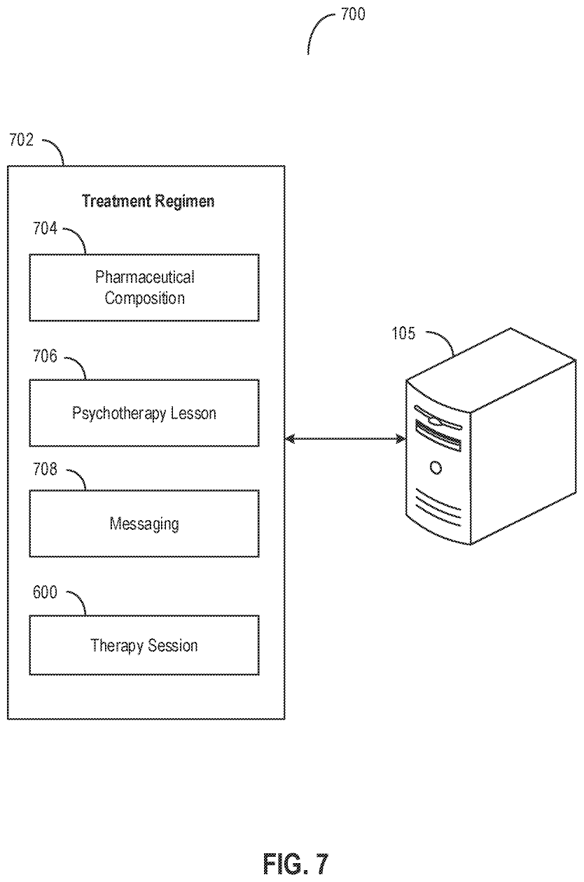

Referring now to FIG. 7, depicted is a block diagram of a process 700 for providing treatment regimen in the system for managing sessions. In some embodiments, the treatment regimen is prescribed based on the subject's MSE score and/or scores from other preliminary evaluations such as subject health questionnaires. In some embodiments, the prescribing comprises communicating the score to a remote server (e.g. a chronic pain treatment server 109) for evaluation by a prescribing health care provider. In some embodiments, the treatment regimen comprises a frequency of conducting therapy session(s) (e.g. once a week, twice a week, three times a week, daily, every other week, etc. . . . ) and an absolute number of times (e.g. 7 times, 10 times, 15 times, 20 times, 70 times, etc. . . . ) to conduct therapy session(s) or a duration of the treatment regimen (e.g. ten days, two weeks, six weeks, etc. . . . ). In some embodiments, the treatment regimen is updated based on the subject's performance in previous therapy sessions. For example, the frequency of the therapy sessions may be reduced after good performance in previous therapy sessions.

The regimen may comprise one or more therapy sessions comprising predetermined numbers of trials, each therapy session having at least 1 to 10000 trials. For example, the treatment regimen may comprise the generation of two short therapy sessions five times per week, or it may comprise the generation of three long therapy sessions two times per week. The treatment regimen may comprise generation of therapy sessions at regular time intervals (for example, anywhere from hourly to once a week). In some embodiments, breaks are incorporated into therapy sessions, wherein a therapy session comprises multiple sets of predetermined numbers of trials, with a break between each set of trials. In some embodiments, feedback is provided to the user during or after therapy sessions. Feedback may comprise information about, for example, the subject's response times, number of correct responses, or the type of stimuli displayed.

In some embodiments, the treatment regimen further comprises the use of a pharmaceutical composition 704, a psychotherapy lesson 706, and/or messaging 708. When used in combination, the therapy sessions 600, the pharmaceutical composition 704, the psychotherapy lessons 706, and/or messaging 708 may serve or function as a synergistic combination therapy for the treatment of chronic pain. The pharmaceutical composition 704 prescribed will depend on the chronic pain disorder being treated. Pharmaceutical compositions 704 known for treating chronic pain disorders include, but are not limited to compositions such as anti-inflammatory compositions, triptans, antiemetics, ergots, neurotoxin injections, calcitonin gene-related peptide (CGRP) inhibitors, anti-depressants, beta-blockers and anti-epileptics.

Whereas the therapy sessions 600 target implicit, cognitive neurological processes of the subject, psychotherapy lessons 706 comprise training on conscious, top-down, or explicit activities of the subject, and therefore may serve or function as a synergistic combination therapy for the treatment of chronic pain. The psychotherapy lessons 706 may comprise training on any behavior-change or insight-based therapeutic activity or skill. The psychotherapy lessons 706 may address impairments in social or behavioral functioning related to chronic pain and/or self-pain enmeshment. For example, the psychotherapy lessons 706 may comprise mindfulness exercises (to reduce attention to pain), self-compassion exercises (to combat self-criticism), or social skills training (to combat other maladaptive behaviors).

Each psychotherapy lesson 706 may comprise a video, text, set of images, audio, haptic feedback, or other content, or combinations thereof. Furthermore, each psychotherapy lesson 706 may have one or more parameters configurable to maximize the effectiveness and impact of the psychotherapy lesson. For example, content of a psychotherapy lesson 706 may be configured to align with the type of chronic pain from which the subject suffers, such as migraine vs. lower back pain.

Messaging 708 may comprise the sending of messages to reinforce psychotherapy lessons 706, said messages delivered to synchronize with the subject's progress through the treatment regimen 702. The messaging 708 may be implemented via short message service (SMS), multimedia message service (MMS), push notifications and the like. The messaging 708 may be delivered periodically, such as daily, weekly, monthly, etc. . . . . The messaging 708 may be derived from a library of pre-generated psychotherapy messages and/or a library of pre-generated engagement (reminder) messages. The messaging 708 may include reminders for the subject to complete the therapy sessions 600, to take the medication 704, and/or to complete the psychotherapy lessons 706 over the course of the treatment regimen 702. The messaging 708 may be personalized based on the subject's activity, adherence, and/or performance in relation to the treatment regimen.

In certain embodiments, the treatment regimen 702 comprises one or more programs that include instructions for intermittently evaluating the subject for one or more symptoms of the chronic pain or self-pain enmeshment disorder being treated, or for co-morbidities or associated symptoms of the chronic pain or self-pain enmeshment disorder being treated. In particular embodiments, the instructions comprise instructions for performing a subject health questionnaire, such as a questionnaire for pain assessment, depression, anxiety, and pain catastrophizing intermittently. Other evaluations that may be related to the treatment include computer proficiency, cognition, self-compassion, and mindfulness. Examples of such tests include, for example, PROMIS pain interference, PROMIS-DSF, PROMIS-ASF, Numerical Rating Scale, Hamilton Depression Rating Scale (HDRS), Pain Catastrophizing Scale, Self-Compassion Scale (SCS), Mobile Device Proficiency Questionnaire (MDPQ), tests that test digit-span forward and back, and letter number sequencing.

Other aspects of the present disclosure are directed to a system to conduct therapy session trials, where each trial includes displaying a self image in a Target location, displaying an other image in an other location, and displaying a stimulus for a stimulus display period, where the stimulus is associated with pain or non-pain. Further, the system may receive a selection signal encoding a Target selection or an Other selection, and may determine a selection status based on the selection signal, where a correct selection comprises (a) the Target selection when the non-pain stimulus is displayed or (b) the Other selection when the pain stimulus is displayed, and an incorrect selection comprises (a) the Target selection when the pain stimulus is displayed or (b) the Other selection when the non-pain stimulus is displayed.

In some implementations, the stimulus of the system may be configured to induce human limbic system activation. Further, the stored program instructions may also comprise determining a response time equal to the time of the receiving the selection signal minus the time of displaying the stimulus. In an embodiment, the one or more therapy sessions are integral to a prescribed treatment regimen.

According to another aspect of the disclosure, the aforementioned prescribed treatment regimen may comprise conducting a sequence of one or more psychotherapy lessons, where each psychotherapy lesson comprises training on conscious behavioral activities. In a further embodiment, the stored program instructions include transmitting one or more messages, where each of the one or more messages are configured to reinforce the therapy session or the psychotherapy lesson, and where each of the one or more messages are synchronized with progress through the treatment regimen.

In further implementations, the stimulus may be selected from the computer-readable memory for display based on a stimulus threshold. The stimulus threshold may be increased when the response time is under a response time threshold of a preceding trial or when a correct to incorrect response ratio of a set of preceding trials exceeds a performance threshold. Also, the stimulus display period may be decreased when the response time is under a response time threshold of a preceding trial or when a correct to incorrect response ratio exceeds a prescribed threshold or exceeds that of a set of preceding trials.

The stimulus of the system may be configured to induce human limbic system activation. Further, the stored program instructions may also comprise determining a response time equal to the time of receiving the selection signal minus the time of displaying the stimulus. In an embodiment, the one or more therapy sessions are integral to a prescribed treatment regimen.

The aforementioned prescribed treatment regimen may comprise conducting a sequence of one or more psychotherapy lessons, where each psychotherapy lesson comprises training on conscious activities. In a further embodiment, the stored program instructions include transmitting one or more messages, where each of the one or more messages are configured to reinforce the therapy session or the psychotherapy lesson, and where each of the one or more messages are synchronized with progress through the treatment regimen.

In an embodiment, the stimulus is selected from the one or more computer-readable memories for display based on a stimulus threshold. The stimulus threshold may be increased when the response time is under a response time threshold of a preceding trial or when a correct to incorrect response ratio of a set of preceding trials exceeds a performance threshold. Also, the stimulus display period may be decreased when the response time is under a response time threshold of a preceding trial or when a correct to incorrect response ratio increases compared to a set of preceding trials.

### B. Network and Computing Environment

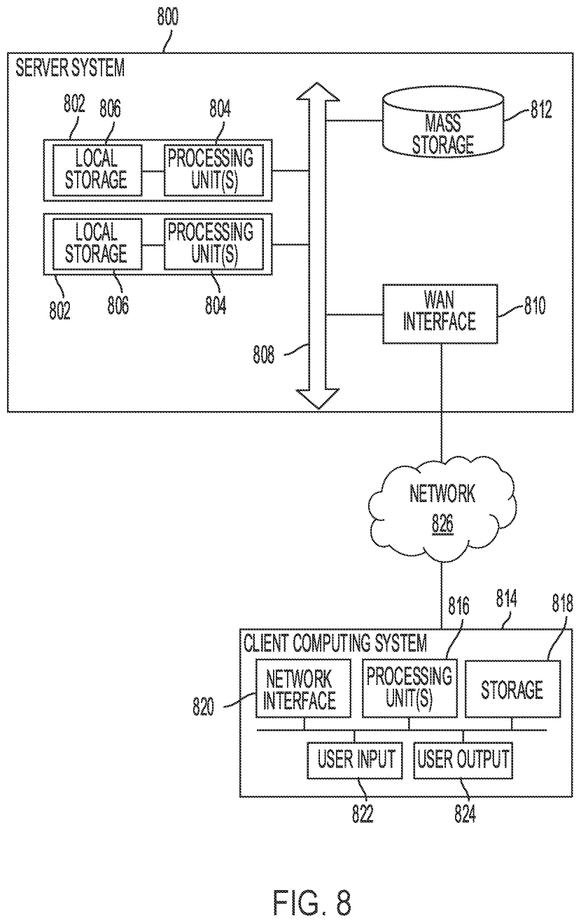

Various operations described herein can be implemented on computer systems. FIG. 8 shows a simplified block diagram of a representative server system 800, client computer system 814, and network 826 usable to implement certain embodiments of the present disclosure. In various embodiments, server system 800 or similar systems can implement services or servers described herein or portions thereof. Client computer system 814 or similar systems can implement clients described herein. The system 100, among others, described herein can be similar to the server system 800. Server system 800 can have a modular design that incorporates a number of modules 802 (e.g., blades in a blade server embodiment); while two modules 802 are shown, any number can be provided. Each module 802 can include processing unit(s) 804 and local storage 806.

Processing unit(s) 804 can include a single processor, which can have one or more cores, or multiple processors. In some embodiments, processing unit(s) 804 can include a general-purpose primary processor as well as one or more special-purpose co-processors such as graphics processors, digital signal processors, or the like. In some embodiments, some or all processing units 804 can be implemented using customized circuits, such as application specific integrated circuits (ASICs) or field programmable gate arrays (FPGAs). In some embodiments, such integrated circuits execute instructions that are stored on the circuit itself. In other embodiments, processing unit(s) 804 can execute instructions stored in local storage 806. Any type of processors in any combination can be included in processing unit(s) 804.

Local storage 806 can include volatile storage media (e.g., DRAM, SRAM, SDRAM, or the like) and/or non-volatile storage media (e.g., magnetic or optical disk, flash memory, or the like). Storage media incorporated in local storage 806 can be fixed, removable, or upgradeable as desired. Local storage 806 can be physically or logically divided into various subunits such as a system memory, a read-only memory (ROM), and a permanent storage device. The system memory can be a read-and-write memory device or a volatile read-and-write memory, such as dynamic random-access memory. The system memory can store some or all of the instructions and data that processing unit(s) 804 need at runtime. The ROM can store static data and instructions that are needed by processing unit(s) 804. The permanent storage device can be a non-volatile read-and-write memory device that can store instructions and data even when module 802 is powered down. The term “storage medium” as used herein includes any medium in which data can be stored indefinitely (subject to overwriting, electrical disturbance, power loss, or the like) and does not include carrier waves and transitory electronic signals propagating wirelessly or over wired connections.

In some embodiments, local storage 806 can store one or more software programs to be executed by processing unit(s) 804, such as an operating system and/or programs implementing various server functions such as functions of the system 100 or any other system described herein, or any other server(s) associated with system 100 or any other system described herein.

“Software” refers generally to sequences of instructions that, when executed by processing unit(s) 804, cause server system 800 (or portions thereof) to perform various operations, thus defining one or more specific machine embodiments that execute and perform the operations of the software programs. The instructions can be stored as firmware residing in read-only memory and/or program code stored in non-volatile storage media that can be read into volatile working memory for execution by processing unit(s) 804. Software can be implemented as a single program or a collection of separate programs or program modules that interact as desired. From local storage 806 (or non-local storage described below), processing unit(s) 804 can retrieve program instructions to execute and data to process in order to execute various operations described above.

In some server systems 800, multiple modules 802 can be interconnected via a bus or other interconnect 808, forming a local area network that supports communication between modules 802 and other components of server system 800. Interconnect 808 can be implemented using various technologies including server racks, hubs, routers, etc.

A wide area network (WAN) interface 810 can provide data communication capability between the local area network (interconnect 808) and the network 826, such as the Internet. Technologies can be used, including wired (e.g., Ethernet, IEEE 802.3 standards) and/or wireless technologies (e.g., Wi-Fi, IEEE 802.11 standards).

In some embodiments, local storage 806 is intended to provide working memory for processing unit(s) 804, providing fast access to programs and/or data to be processed while reducing traffic on interconnect 808. Storage for larger quantities of data can be provided on the local area network by one or more mass storage subsystems 812 that can be connected to interconnect 808. Mass storage subsystem 812 can be based on magnetic, optical, semiconductor, or other data storage media. Direct attached storage, storage area networks, network-attached storage, and the like can be used. Any data stores or other collections of data described herein as being produced, consumed, or maintained by a service or server can be stored in mass storage subsystem 812. In some embodiments, additional data storage resources may be accessible via WAN interface 810 (potentially with increased latency).

Server system 800 can operate in response to requests received via WAN interface 810. For example, one of modules 802 can implement a supervisory function and assign discrete tasks to other modules 802 in response to received requests. Work allocation techniques can be used. As requests are processed, results can be returned to the requester via WAN interface 810. Such operation can generally be automated. Further, in some embodiments, WAN interface 810 can connect multiple server systems 800 to each other, providing scalable systems capable of managing high volumes of activity. Other techniques for managing server systems and server farms (collections of server systems that cooperate) can be used, including dynamic resource allocation and reallocation.

Server system 800 can interact with various user-owned or user-operated devices via a wide-area network such as the Internet. An example of a user-operated device is shown in FIG. 8 as client computing system 814. Client computing system 814 can be implemented, for example, as a consumer device such as a smartphone, other mobile phone, tablet computer, wearable computing device (e.g., smart watch, eyeglasses), desktop computer, laptop computer, and so on.

For example, client computing system 814 can communicate via WAN interface 810. Client computing system 814 can include computer components such as processing unit(s) 816, storage device 818, network interface 820, user input device 822, and user output device 824. Client computing system 814 can be a computing device implemented in a variety of form factors, such as a desktop computer, laptop computer, tablet computer, smartphone, other mobile computing device, wearable computing device, or the like.

Processor 816 and storage device 818 can be similar to processing unit(s) 804 and local storage 806 described above. Suitable devices can be selected based on the demands to be placed on client computing system 814; for example, client computing system 814 can be implemented as a “thin” client with limited processing capability or as a high-powered computing device. Client computing system 814 can be provisioned with program code executable by processing unit(s) 816 to enable various interactions with server system 800.

Network interface 820 can provide a connection to the network 826, such as a wide area network (e.g., the Internet) to which WAN interface 810 of server system 800 is also connected. In various embodiments, network interface 820 can include a wired interface (e.g., Ethernet) and/or a wireless interface implementing various RF data communication standards such as Wi-Fi, Bluetooth, or cellular data network standards (e.g., 3G, 4G, LTE, etc.).

User input device 822 can include any device (or devices) via which a user can provide signals to client computing system 814; client computing system 814 can interpret the signals as indicative of particular user requests or information. In various embodiments, user input device 822 can include any or all of a keyboard, touch pad, touch screen, mouse or other pointing device, scroll wheel, click wheel, dial, button, switch, keypad, microphone, and so on.

User output device 824 can include any device via which client computing system 814 can provide information to a user. For example, user output device 824 can include display-to-display images generated by or delivered to client computing system 814. The display can incorporate various image generation technologies, e.g., a liquid crystal display (LCD), light-emitting diode (LED) including organic light-emitting diodes (OLED), projection system, cathode ray tube (CRT), or the like, together with supporting electronics (e.g., digital-to-analog or analog-to-digital converters, signal processors, or the like). Some embodiments can include a device such as a touchscreen that function as both input and output device. In some embodiments, other user output devices 824 can be provided in addition to or instead of a display. Examples include indicator lights, speakers, tactile “display” devices, printers, and so on.

Some embodiments include electronic components, such as microprocessors, storage and memory that store computer program instructions in a computer readable storage medium. Many of the features described in this specification can be implemented as processes that are specified as a set of program instructions encoded on a computer readable storage medium. When these program instructions are executed by one or more processing units, they cause the processing unit(s) to perform various operations indicated in the program instructions. Examples of program instructions or computer code include machine code, such as is produced by a compiler, and files including higher-level code that are executed by a computer, an electronic component, or a microprocessor using an interpreter. Through suitable programming, processing unit(s) 804 and 816 can provide various functionality for server system 800 and client computing system 814, including any of the functionality described herein as being performed by a server or client, or other functionality.

It will be appreciated that server system 800 and client computing system 814 are illustrative and that variations and modifications are possible. Computer systems used in connection with embodiments of the present disclosure can have other capabilities not specifically described here. Further, while server system 800 and client computing system 814 are described with reference to particular blocks, it is to be understood that these blocks are defined for convenience of description and are not intended to imply a particular physical arrangement of component parts. For instance, different blocks can be but need not be located in the same facility, in the same server rack, or on the same motherboard. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Embodiments of the present disclosure can be realized in a variety of apparatus including electronic devices implemented using any combination of circuitry and software.

While the disclosure has been described with respect to specific embodiments, one skilled in the art will recognize that numerous modifications are possible. Embodiments of the disclosure can be realized using a variety of computer systems and communication technologies including but not limited to specific examples described herein. Embodiments of the present disclosure can be realized using any combination of dedicated components and/or programmable processors and/or other programmable devices. The various processes described herein can be implemented on the same processor or different processors in any combination. Where components are described as being configured to perform certain operations, such configuration can be accomplished; e.g., by designing electronic circuits to perform the operation, by programming programmable electronic circuits (such as microprocessors) to perform the operation, or any combination thereof. Further, while the embodiments described above may make reference to specific hardware and software components, those skilled in the art will appreciate that different combinations of hardware and/or software components may also be used and that particular operations described as being implemented in hardware might also be implemented in software or vice versa.

Computer programs incorporating various features of the present disclosure may be encoded and stored on various computer readable storage media; suitable media include magnetic disk or tape, optical storage media such as compact disk (CD) or digital versatile disk (DVD), flash memory, and other non-transitory media. Computer readable media encoded with the program code may be packaged with a compatible electronic device, or the program code may be provided separately from electronic devices (e.g., via Internet download or as a separately packaged computer-readable storage medium).

Thus, although the disclosure has been described with respect to specific embodiments, it will be appreciated that the disclosure is intended to cover all modifications and equivalents within the scope of the following claims.

What is claimed is:

1. A system for managing sessions for subjects, comprising:
a computing system having one or more processors coupled with memory, the computing system configured to:
identify, using a user profile of a subject maintained on a database, a condition of the subject to be addressed and a plurality of images of expressions associated with the subject;
select, for a first session trial for subject, (i) a first image from the plurality of images of expressions associated with the subject, (ii) a second image associated with another subject, and (iii) a third image corresponding to one of a plurality of types for the condition;
determine a presentation parameter for the first session trial based on the user profile;
provide, for presentation of the first session trial to the subject, (i) the first image, (ii) the second image, and (iii) the third image, in accordance with the presentation parameter;
receive, from the subject, a response identifying an association of the third image with one of the first image or the second image;
determine a performance metric of the subject for the first session trial based on the association identified in the response and a type of the plurality of types corresponding to the third image; and
update, using the performance metric, the presentation parameter to modify the presentation for a second session trial and the user profile in relation to the condition.

2. The system of claim 1, wherein the computing system is further configured to:
select the third image corresponding to an associative type for the condition; and
determine, responsive to the association of the third image as with the second image, the performance metric to indicate the response as a correct selection.

3. The system of claim 1, wherein the computing system is further configured to:
select the third image corresponding to an associative type for the condition; and
determine, responsive to the association of the third image as with the first image, the performance metric to indicate the response as an incorrect selection.

4. The system of claim 1, wherein the computing system is further configured to select, from a plurality of images of expressions associated with one or more subjects, the third image based on an intensity level for the first session trial.

5. The system of claim 1, wherein the computing system is further configured to determine the presentation parameter to define a length of the presentation of the third image on a display, using a second performance metric of a third session trial.

6. The system of claim 1, wherein the computing system is further configured to determine the presentation parameter to define a location of the presentation of the third image on a display relative to the first image and the second image to increase likelihood of a correct selection.

7. The system of claim 1, wherein the computing system is further configured to determine the performance metric based on a comparison between (i) a time elapsed between the presentation of the third image and the receipt of the response and (ii) a threshold time for the third image.

8. The system of claim 1, wherein the computing system is further configured to provide, responsive to receiving the response, for presentation to the subject, an indication of the response as one of a correct selection or an incorrect selection based on the association.

9. The system of claim 1, wherein the computing system is further configured to provide, via a display, a graphical user interface to associate the third image with one of the first image or the second image.

10. The system of claim 1, wherein the condition is a condition associated with chronic pain.

11. The system of claim 10, wherein the condition associated with chronic pain comprises one or more of: arthritis, migraine, fibromyalgia, back pain, Lyme disease, endometriosis, repetitive stress injuries, irritable bowel syndrome, inflammatory bowel disease or cancer pain.

12. The system of claim 10, wherein the user is on a pain relief medication to address the chronic pain, at least in partial concurrence with a plurality of session trials.

13. A method of managing sessions for subjects, comprising:
identifying, by a computing system, using a user profile of a subject maintained on a database, a condition of the subject to be addressed and a plurality of images of expressions associated with the subject;
selecting, by the computing system, for a first session trial for subject, (i) a first image from the plurality of images of expressions associated with the subject, (ii) a second image associated with another subject, and (iii) a third image corresponding to one of a plurality of types for the condition;
determining, by the computing system, a presentation parameter for the first session trial based on the user profile;
providing, by the computing system, for presentation of the first session trial to the subject, (i) the first image, (ii) the second image, and (iii) the third image, in accordance with the presentation parameter;
receiving, by the computing system, from the subject, a response identifying an association of the third image with one of the first image or the second image;
determining, by the computing system, a performance metric of the subject for the first session trial based on the association identified in the response and a type of the plurality of types corresponding to the third image; and
updating, by the computing system, using the performance metric, the presentation parameter to modify the presentation and the user profile to identify in relation to the condition.

14. The method of claim 13, further comprising selecting, by the computing system, the third image corresponding to an associative type for the condition; and
wherein determining the performance metric further comprises determining, responsive to the association of the third image as with the second image, the performance metric to indicate the response as a correct selection.

15. The method of claim 13, further comprising selecting, by the computing system, the third image corresponding to an associative type for the condition; and
wherein determining the performance metric further comprises determining, responsive to the association of the third image as with the first image, the performance metric to indicate the response as an incorrect selection.

16. The method of claim 13, further comprising selecting, by the computing system, from a plurality of images of expressions associated with one or more subjects, the third image based on an intensity level for the first session trial.

17. The method of claim 13, wherein determining the presentation parameter further comprises determining the presentation parameter to define a length of the presentation of the third image on a display, using a second performance metric of a third session trial.

18. The method of claim 13, wherein determining the presentation parameter further comprises determining the presentation parameter to define a location of the presentation of the third image on a display relative to the first image and the second image to increase likelihood of a correct selection.

19. The method of claim 13, wherein determining the presentation parameter further comprises determining the presentation parameter based on a comparison between (i) a time elapsed between the presentation of the third image and the receipt of the response and (ii) a threshold time for the third image.

20. The method of claim 13, further comprising providing, by the computing system, responsive to receiving the response, for presentation to the subject, an indication of the response as one of a correct selection or an incorrect selection based on the association.

21. The method of claim 13, further comprising providing, by the computing systems, via a display, a graphical user interface to associate the third image with one of the first image or the second image.

22. The method of claim 13, wherein the condition is a condition associated with chronic pain.

23. The method of claim 22, wherein the condition associated with chronic pain comprises one or more of: arthritis, migraine, fibromyalgia, back pain, Lyme disease, endometriosis, repetitive stress injuries, irritable bowel syndrome, inflammatory bowel disease or cancer pain.

24. The method of claim 22, wherein the user is on a pain relief medication to address the chronic pain, at least in partial concurrence with a plurality of session trials.
